(19) United States
(12) Patent Application Publication
(10) Pub. No.: US 2025/0041419 A1
(43) Pub. Date: Feb. 6, 2025

(54) Modified Cytotoxic T Cells and Methods of Use Thereof

(71) Applicant: Cue Biopharma, Inc., Boston, MA (US)

(72) Inventor: Anish Suri, Boston, MA (US)

(21) Appl. No.: 18/927,002

(22) Filed: Oct. 25, 2024

Related U.S. Application Data
(63) Continuation of application No. PCT/US2023/066174, filed on Apr. 25, 2023.
(60) Provisional application No. 63/336,041, filed on Apr. 28, 2022.

(51) Int. Cl. A61K 39/00; C07K 14/725; C07K 16/08

(52) U.S. Cl. CPC A61K 39/464838; A61K 39/4611; A61K 39/4631; C07K 14/7051; C07K 16/084

(57) ABSTRACT

The present disclosure provides modified cytotoxic T cells (mCTLs), where the mCTLs comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2); and b) one or more nucleic acids comprising nucleotide sequences encoding a chimeric antigen receptor (CAR), where the CAR comprises an antigen-binding domain specific for a cancer-associated antigen. The present disclosure provides methods of producing the mCTLs. The present disclosure provides methods of treating cancer, comprising administering the mCTLs to an individual in need thereof.

### CROSS-REFERENCE

This application claims the benefit of U.S. Provisional Patent Application No. 63/336,041, filed Apr. 28, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “CUEB-150WO_SEQ_LIST” created on Apr. 23, 2023 and having a size of 136,686 bytes. The contents of the Sequence Listing XML are incorporated by reference herein in their entirety.

### INTRODUCTION

Cell therapy products include T cells that are modified to express proteins that bind to proteins on cancer cells. For example, “CAR-T cells” comprise a chimeric antigen receptor (“CAR”) that targets tumor-associated antigens. CAR-T cells can kill tumor cells and have been used successfully to treat cancers. For example, CAR-T cells targeting CD19, BCMA, CD30, CD22, or CD20 have shown significant activity in clinical studies. CAR-T therapies can suffer from drawbacks, however. Due to the large number of CAR-T cells that are administered, patients typically require a chemotherapy regimen for lymphodepletion prior to administration of the CAR-T cells. Also, to maintain therapeutic levels of CAR-T cells following administration, patients also may require treatment with aldesleukin (Proleukin®), which can result in serious adverse side effects. Further, some patients may not achieve their desired or optimal result due to suboptimal expansion or persistence of CAR-T cells in vivo.

### SUMMARY

This disclosure includes disclosure of modified cytotoxic T cells (mCTLs), where the mCTLs may comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) (an “HPV E7₁₁₋₂₀ TCR”) or YMLDLQPET (SEQ ID NO:2) (an “HPV E7₁₁₋₁₉ TCR”) (collectively referred to as an “HPV E7_{11-19/20} TCR”); and b) one or more nucleic acids comprising nucleotide sequences encoding an antigen binding polypeptide, e.g., a CARs or other polypeptide that comprises an antigen-binding domain specific for a cancer-associated antigen. The present disclosure also includes methods of producing the mCTLs. The present disclosure also provides methods of treating cancer, comprising administering the mCTLs to an individual in need thereof. This disclosure also includes disclosure of mCTLs having a TCR other than an HPV E7_{11-19/20} TCR, as well as a disclosure of modified cells other than T cells, e.g., NK cells, which are modified to present both a TCR and a CAR or other cancer-associated antigen-binding domain.

### BRIEF DESCRIPTION OF THE DRAWINGS

FIG. 1 is a schematic depiction of generation and use of mCTLs according to the present disclosure.

FIG. 2A-2D provide amino acid sequences of exemplary TCR alpha and beta chains (SEQ ID Nos: 19-22).

FIG. 3A-3D provide amino acid sequences of a polypeptide of a T-cell modulatory polypeptide (TMP) (SEQ ID NO:15) (FIG. 3A) and amino acid sequences of the components of the polypeptide (SEQ ID Nos: 5, 8 and 10) (FIG. 3B-3D).

FIG. 4A-4B provides an amino acid sequence of a polypeptide of a TM (SEQ ID NO:14) (FIG. 4A) and the amino acid sequence of a β2M polypeptide (SEQ ID NO:4) (FIG. 4B).

FIG. 5 provides sequence identifiers for certain amino acid sequences noted in the specification and figures (SEQ ID Nos: 1-18).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4+ cells), cytotoxic T-cells (CD8+ cells), T-regulatory cells (Treg), and NK-T cells.

The terms “expression construct,” or “DNA construct” are used interchangeably herein to refer to a DNA molecule comprising a vector and at least one insert.

As used herein, the term “affinity” refers to the equilibrium constant for the reversible binding of two agents (e.g., an antibody and an antigen) and is expressed as a dissociation constant (K_{D}). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1,000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments. Unless otherwise indicated herein, when the word “about” is used in reference to a numeric value, it means a range of ±10% of the stated numeric value, e.g., “about 10” means a value from 9 to 11.

The term “binding,” as used herein (e.g., with reference to binding of a T cell modulatory polypeptide (“TMP”) to a polypeptide (e.g., a T-cell receptor) on a T cell; or with reference to binding of an antigen-binding polypeptide present in a CAR to an antigen such as a cancer-associated antigen), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. Non-covalent binding interactions are generally characterized by a dissociation constant (K_{D}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of non-covalent binding, increased binding affinity being correlated with a lower K_{D}. “Specific binding” generally refers to binding with an affinity of at least about 10⁻⁷ M or greater, e.g., 5×10⁻⁷ M, 10⁻⁸ M, 5×10⁻⁸ M, 10⁻⁹ M, and greater. “Non-specific binding” generally refers to binding (e.g., the binding of a ligand to a moiety other than its designated binding site or receptor) with an affinity of less than about 10⁻⁷ M (e.g., binding with an affinity of 10⁶ M, 10⁵ M, 10⁴ M). However, in some contexts, e.g., binding between a TCR and a peptide/MHC complex, “specific binding” can be in the range of from 1 μM to 100 μM, or from 100 μM to 1 mM. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC. Unless otherwise indicated, when used herein the term “substantially” means “wholly or largely but not wholly”. As another example, a “substantially homogeneous population” means a population that is wholly homogeneous or largely but not wholly homogeneous.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “humanized immunoglobulin” as used herein refers to an immunoglobulin comprising portions of immunoglobulins of different origin, wherein at least one portion comprises amino acid sequences of human origin. For example, the humanized antibody can comprise portions derived from an immunoglobulin of nonhuman origin with the requisite specificity, such as a mouse, and from immunoglobulin sequences of human origin (e.g., chimeric immunoglobulin), joined together chemically by conventional techniques (e.g., synthetic) or prepared as a contiguous polypeptide using genetic engineering techniques (e.g., DNA encoding the protein portions of the chimeric antibody can be expressed to produce a contiguous polypeptide chain). Another example of a humanized immunoglobulin is an immunoglobulin containing one or more immunoglobulin chains comprising a complementarity-determining region (CDR) derived from an antibody of nonhuman origin and a framework region derived from a light and/or heavy chain of human origin (e.g., CDR-grafted antibodies with or without framework changes). Chimeric or CDR-grafted single chain antibodies are also encompassed by the term humanized immunoglobulin. See, e.g., U.S. Pat. No. 4,816,567; European Patent No. 0,125,023 B1; U.S. Pat. No. 4,816,397; European Patent No. 0,120,694 B1; WO 86/01533; European Patent No. 0,194,276 B1; U.S. Pat. No. 5,225,539; European Patent No. 0,239,400 B1; and European Patent Application No. 0,519.596 A1. See also, U.S. Pat. Nos. 4,946,778; 5,476,786; and Bird et al. (1988) Science 242:423, regarding single chain antibodies.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8 (10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and—binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2. IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the V_{H} and V_{L} domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues, which encompass the CDRs, as defined by each of the above cited references are set forth below in Table 1 as a comparison.

TABLE 1
CDR Definitions

| | Kabat¹ | Chothia² | MacCallum³ |
|---|---|---|---|
| V_{H} CDR-1 | 31-35 | 26-32 | 30-35 |
| V_{H} CDR-2 | 50-65 | 53-55 | 47-58 |
| V_{H} CDR-3 | 95-102 | 96-101 | 93-101 |
| V_{L} CDR-1 | 24-34 | 26-32 | 30-36 |
| V_{L} CDR-2 | 50-56 | 50-52 | 46-55 |
| V_{L} CDR-3 | 89-97 | 91-96 | 89-96 |

¹Residue numbering follows the nomenclature of Kabat et al., 1991, supra
²Residue numbering follows the nomenclature of Chothia et al., supra
³Residue numbering follows the nomenclature of MacCallum et al., supra

As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

As used herein, the term “framework,” when used in reference to an antibody variable region, is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a modified T cell” includes a plurality of such T cells and reference to “the T-cell modulatory polypeptide” or TMP includes reference to one or more T-cell modulatory polypeptides or TMPs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

### DETAILED DESCRIPTION

The present disclosure provides modified cytotoxic T cells (mCTLs), where the mCTLs may comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) (an “HPV E7₁₁₋₂₀ TCR”) or YMLDLQPET (SEQ ID NO:2) (an “HPV E7₁₁₋₁₉ TCR”) (collectively referred to as an “HPV E7_{11-19/20} TCR”); and b) one or more nucleic acids comprising nucleotide sequences encoding an antigen binding polypeptide, e.g., a CARs or other polypeptide that comprises an antigen-binding domain specific for a cancer-associated antigen. The present disclosure also includes methods of producing the mCTLs. The present disclosure also provides methods of treating cancer, comprising administering the mCTLs to an individual in need thereof. This disclosure also includes disclosure of mCTLs having a TCR other than an HPV E7_{11-19/20} TCR, as well as a disclosure of modified cells other than T cells, e.g., NK cells, which are modified to present both a TCR and a CAR or other cancer-associated antigen-binding domain.

### Modified Cytotoxic T Cells

The present disclosure provides modified cytotoxic T cells (mCTLs), where the mCTLs comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO: 2); and b) one or more nucleic acids comprising nucleotide sequences encoding an antigen binding polypeptide, e.g., a CAR or other polypeptide that comprises an antigen-binding domain specific for a cancer-associated antigen.

A T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2) is referred to herein as a “HPV E7_{11-19/20} TCR”.

The present disclosure provides methods of producing the mCTLs comprising an HPV E7_{11-19/20} TCR. The present disclosure provides methods of treating cancer, comprising administering the mCTLs comprising an HPV E7_{11-19/20} TCR to an individual in need thereof. The present disclosure also provides mCTLs comprising an HPV E7_{11-19/20} TCR, and an antigen binding polypeptide, e.g., a CAR or other polypeptide that comprise an antigen-binding domain specific for a cancer-associated antigen.

The present disclosure thus provides mCTLs, where the mCTLs comprise: a) one or more nucleic acids comprising nucleotide sequences encoding an HPV E7_{11-19/20} TCR; and b) one or more nucleic acids comprising nucleotide sequences encoding a CAR, where the CAR comprises an antigen-binding domain specific for a cancer-associated antigen.

The present disclosure thus provides mCTLs, where the mCTLs comprise: a) an HPV E7_{11-19/2011-19/20} TCR; and b) a CAR, where the CAR comprises an antigen-binding domain specific for a cancer-associated antigen.

Although the mCTLs are illustrated herein as having an HPV E7_{11-19/20} TCR, i.e., a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO: 2), it will be understood that the mCTLs could have an alternative TCR that is specific for a different peptide, in which case a TMP presenting a peptide for which the alternative TCR is specific would be used. Likewise, the mCTLs are illustrated as comprising CARs, but the mCTLs can be other cell therapy products that comprise an antigen-binding domain for a cancer-associated antigen, wherein the antigen-binding domain is other than a CAR. mCTLs comprising a TCR specific for a different peptide and/or an antigen-binding domain other than a CAR are expressly contemplated by this disclosure. Likewise, while the disclosure is directed primarily to modified cytotoxic T cells (mCTLs) such as CAR-T cells, it will be understood that other types of cytotoxic cells could be modified using the disclosure herein, e.g., NK cells, macrophages; ILC (innate lymphoid cells), which have a natural or modified antigen-binding domains. T cells can include classical T cells (alpha beta receptor expressing), or T cells expressing gamma/delta TCRs; CD8 and CD4 phenotypes; MAIT T cells restricted to MR-1 non-classical HLA, killer T cells and killer innate-like T cells.

In some cases, the mCTLs are in vitro. An in vitro composition of the present disclosure can comprise a population of T cells that may contain T cells other than the mCTLs. Such cells are referred to as “unmodified T cells.” Thus, an in vitro composition of the present disclosure can be a heterogeneous population comprising mCTLs and unmodified T cells. In some cases, from 1% to 20% of the total number of T cells in the composition are mCTLs. In some cases, from 1% to 5%, from 5% to 10%, from 10% to 15%, or from 15% to 20% of the total number of T cells in the composition are mCTLs. In some cases, at least 20%, at least 25%, at least 30%, at least 35%, at least 40%, at least 50%, at least 60%, at least 70%, at least 75%, at least 80%, at least 85%, at least 90%, at least 95%, at least 98%, at least 99%, or more than 99%, of the total number of T cells in the composition are mCTLs. In some cases, from 20% to 30%, from 30% to 40%, from 40% to 50%, from 50% to 60%, from 60% to 70%, from 70% to 80%, from 80% to 90%, or from 90% to 100%, of the total number of T cells in the composition are mCTLs. In some cases, therefore, the population of T cells in the composition is a substantially homogeneous population of mCTLs. As noted above, a “substantially homogeneous population” means a population that is wholly homogeneous or largely but not wholly homogeneous.

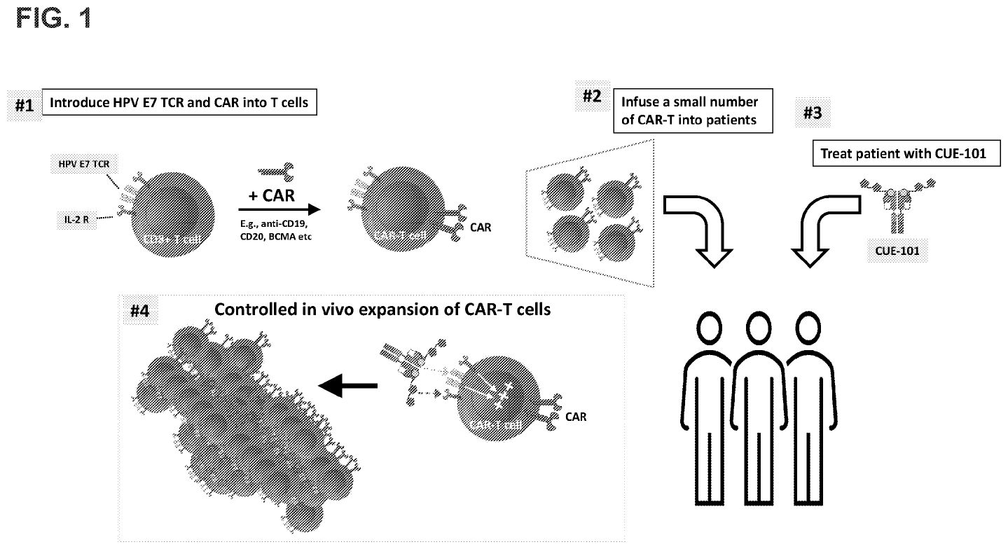

An exemplary use of an in vivo composition comprising mCTLs is depicted schematically in FIG. 1. An in vitro cell population is modified to express a CAR specific for a target cancer-associated antigen and to express a heterologous HPV E7_{11-19/20} TCR. The in vitro composition comprising target modified T cells is then be administered to an individual in need thereof, e.g., an individual having a cancer that expresses the target cancer-associated antigen. A T-cell modulatory polypeptide (TMP) that comprises the HPV E7 peptide comprising the amino acid sequence YMLDLQPETT (SEQ ID NO:1) or YMLDLQPET (SEQ ID NO:2) can also be administered to the individual. The TMP comprises an immunomodulatory polypeptide that provides for activation of T cells comprising HPV E7_{11-19/20} TCRs that bind the HPV E7 peptide present in the TMP. The modified target T cells (e.g., mCTLs) will target cancer cells expressing the cancer-associated antigen to which the CAR present. The TMP will activate the modified target T cells by binding to TCRs present on the modified target T cells. Activation of a modified target can provide one or both of (i) increased direct killing of a cancer cell; and (ii) increased secretion of cytokines that promote anti-cancer activity of resident immune cells. The TMP can increase in vivo proliferation of the mCTLs, thereby reducing the number of mCTLs required to be administered for anti-cancer therapy, and also reducing or substantially eliminating the need for lymphodepleting chemotherapy before administration of the mCTLs, and/or reducing or substantially eliminating the need for treatment with recombinantly produced IL-2, e.g., aldesleukin (Proleukin®), following administration of CAR-T cells.

### Chimeric Antigen Receptor

As noted above, a modified T cell is modified to express a CAR specific for a cancer-associated antigen. A CAR generally comprises: a) an extracellular domain comprising an antigen-binding domain (antigen-binding polypeptide); b) a transmembrane region; and c) a cytoplasmic domain comprising an intracellular signaling domain (intracellular signaling polypeptide). In some cases, a CAR comprises: a) an extracellular domain comprising the antigen-binding domain; b) a transmembrane region; and c) a cytoplasmic domain comprising: i) one or more co-stimulatory polypeptides; and ii) an intracellular signaling domain. In some cases, a CAR comprises hinge region between the extracellular antigen-binding domain and the transmembrane domain. Thus, in some cases, a CAR comprises: a) an extracellular domain comprising the antigen-binding domain; b) a hinge region; c) a transmembrane region; and d) a cytoplasmic domain comprising an intracellular signaling domain. In some cases, a CAR comprises: a) an extracellular domain comprising the antigen-binding domain; b) a hinge region; c) a transmembrane region; and d) a cytoplasmic domain comprising: i) one or more co-stimulatory polypeptides; and ii) an intracellular signaling domain.

Exemplary CAR structures are known in the art (See e.g., WO 2009/091826; US 20130287748; WO 2015/142675; WO 2014/055657; WO 2015/090229; and U.S. Pat. No. 9,587,020.

In some cases, a CAR is a single polypeptide chain. In some cases, a CAR comprises two polypeptide chains. Generally, any CAR structure known to those skilled in the art may be used to modify T cells in order to prepare compositions as disclosed herein.

CARs specific for a variety of tumor antigens are known in the art; for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J. Natl Cancer Inst (2014) 107(1):364), carbonic anhydrase IX-specific CARs (Lamers et al., Biochem Soc Trans (2016) 44(3):951-959), folate receptor-α (FR-α)-specific CARs (Kershaw et al., Clin Cancer Res (2006) 12(20):6106-6015), HER2-specific CARs (Ahmed et al., J Clin Oncol (2015) 33(15) 1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL-13Rα2-specific CARs (Brown et al., Clin Cancer Res (2015) 21(18):4062-4072), ganglioside GD2-specific CARs (Louis et al., Blood (2011) 118(23):6050-6056; Caruana et al., Nat Med (2015) 21(5):524-529; Yu et al. (2018) J. Hematol. Oncol. 11:1), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2): 154-166), mesothelin (MSLN)-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta™) and Tisagenlecleucel (Kymriah™). See also, Li et al., J Hematol and Oncol (2018) 11:22, reviewing clinical trials of tumor-specific CARs; Heyman and Yan (2019) Cancers 11:pii:E191; Baybutt et al. (2019) Clin. Pharmacol. Ther. 105:71.

For example, CAR-T therapies include Yescarta® (axicabtagene ciloleucel) is a CAR comprising a scFv that binds CD19. As further examples, Tecartus® (brexucabtagene autoleucel) is a CAR comprising a scFv that binds CD19; Kymriah® (tisagenleucleucel) is a CAR comprising a scFv that binds CD19; and Abecma® (idecabtagene vicleucel) comprising a scFv that binds BCMA; and Breyanzi® (lisocabtagene maraleucel) is a CAR comprising a scFv that binds CD19.

### Antigen-Binding Domain

As noted above, a CAR comprises an extracellular domain comprising an antigen-binding domain. The antigen-binding domain present in a CAR can be any antigen-binding polypeptide, a wide variety of which are known in the art. In some instances, the antigen-binding domain is a single chain Fv (scFv). Other antibody-based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable. In some cases, the antigen-binding domain is a nanobody.

In some cases, the antigen bound by the antigen-binding domain of a CAR is selected from: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a melanoma antigen recognized by T-cells (melanA/MART1) polypeptide, a Ras polypeptide, a gp100 polypeptide, a proteinase3 (PR1) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B713; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, or a Fos-related antigen-1 (FOSL) polypeptide. In some cases, the antigen is a human papilloma virus (HPV) antigen. In some cases, the antigen is an alpha-feto protein (AFP) antigen. In some cases, the antigen is a Wilms tumor-1 (WT1) antigen.

The antigen-binding polypeptide of a CAR can bind any of a variety of cancer-associated antigens, including, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), B-cell maturation antigen (BCMA), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein (AFP), BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor 1 receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

In some cases, the cancer-associated antigen bound by the antigen-binding polypeptide of a CAR is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRvIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2. In some cases, the cancer-associated antigen is BCMA. In some cases, the cancer-associated antigen is MUC1. In some cases, the cancer-associated antigen is CD19. In some cases, the cancer-associated antigen is AFP.

VH and VL amino acid sequences of various cancer-associated antigen-binding antibodies are known in the art, as arc the light chain and heavy chain CDRs of such antibodies. See, e.g., Ling et al. (2018) Frontiers Immunol. 9:469; WO 2005/012493; US 2019/0119375; US 2013/0066055. The following are non-limiting examples of antibodies that bind cancer-associated antigens.

### 1) Anti-Her2

In some cases, an anti-Her2 antibody comprises: a) a light chain comprising an amino acid sequence having at least 90%, at least 95%, at least 98%, at least 99%, or 100%, amino acid sequence identity to the following amino acid sequence:

DIQMTQSPSSLSASVGDRVTITCRASQDVNTAVAWYQQKPGKAPKLLIYSASFLY SGVPSRFSGSRSGTDFTLTISSLQPEDFATYYCQQHYTTPPTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:25); and b) a heavy chain comprising an amino acid sequence having at least 90%, at least 95%, at least 98%, at least 99%, or 100%, amino acid sequence identity to the following amino acid sequence:

(SEQ ID NO: 26)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some cases, an anti-Her2 antibody comprises a light chain variable region (VL) present in the light chain amino acid sequence provided above; and a heavy chain variable region (VH) present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:27); and b) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:28). In some cases, an anti-Her2 antibody comprises, in order from N-terminus to C-terminus: a) a VH comprising an amino acid sequence having at least 90%, at least 95%, at least 98%, at least 99%, or 100%, amino acid sequence identity to the amino acid sequence: EVQLVESGGGLVQPGGSLRLSCAASGFNIKDTYIHWVRQAPGKGLEWVARIYPTNGYTRYADS VKGRFTISADTSKNTAYLQMNSLRAEDTAVYYCSRWGGDGFYAMDYWGQGTLVTVSS (SEQ ID NO:28); b) a linker; and c) a VL comprising an amino acid sequence having at least 90%, at least 95%, at least 98%, at least 99%, or 100%, amino acid sequence identity to the amino acid sequence: DIQMTQSPSSLSASVGDRVTITCRASQDVNTAVAWYQQKPGKAPKLLIYSASFLYSGVPSRFSGS RSGTDFTLTISSLQPEDFATYYCQQHYTTPPTFGQGTKVEIK (SEQ ID NO:27). Suitable linkers are described elsewhere herein and include, e.g., (GGGGS)n (SEQ ID NO:29), where n is an integer from 1 to 10 (e.g., 1, 2, 3, 4, 5, 6, 7, 8, 9, or 10).

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-Her2 antibody can comprise a VL CDR1 having the amino acid sequence RASQDVNTAVA (SEQ ID NO:30); a VL CDR2 having the amino acid sequence SASFLY (SEQ ID NO:31); a VL CDR3 having the amino acid sequence QQHYTTPP (SEQ ID NO:32); a VH CDR1 having the amino acid sequence GFNIKDTY (SEQ ID NO:33); a VH CDR2 having the amino acid sequence IYPTNGYT (SEQ ID NO:34); and a VH CDR3 having the amino acid sequence SRWGGDGFYAMDY (SEQ ID NO:35).

In some cases, an anti-Her2 antibody is a scFv antibody. For example, an anti-Her2 scFv can comprise an amino acid sequence having at least 90%, at least 95%, at least 98%, at least 99%, or 100%, amino acid sequence identity to the following amino acid sequence:

(SEQ ID NO: 36)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSL
SASVGDRVTITCRASQDVNTAVAWYQQKPGKAPKLLIYSASFLYSGVPS
RFSGSRSGTDFTLTISSLQPEDFATYYCQQHYTTPPTFGQGTKVEIK.

As another example, in some cases, an anti-Her2 antibody comprises: a) a light chain variable region (VL) comprising an amino acid sequence having at least 90%, at least 95%, at least 98%, at least 99%, or 100%, amino acid sequence identity to the following amino acid sequence:

DIQMTQSPSSLSASVGDRVTITCKASQDVSIGVAWYQQKPGKAPKLLIYSASYRY TGVPSRFSGSGSGTDFTLTISSLQPEDFATYYCQQYYIYPYTFGQGTKVEIKRTVAAPSVFIFPPSD EQLKSGTASVVCLLNNFYPREAKVQWKVDNALQSGNSQESVTEQDSKDSTYSLSSTLTLSKAD YEKHKVYACEVTHQGLSSPVTKSFNRGEC (SEQ ID NO:37); and b) a heavy chain variable region (VH) comprising an amino acid sequence having at least 90%, at least 95%, at least 98%, at least 99%, or 100%, amino acid sequence identity to the following amino acid sequence:

(SEQ ID NO: 38)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSKLTVDKSRWQQGNVFSCSVMHEALHNHYTQK
SLSLSPG.

In some cases, an anti-Her2 antibody comprises a VL present in the light chain amino acid sequence provided above; and a VH present in the heavy chain amino acid sequence provided above. For example, an anti-Her2 antibody can comprise: a) a VL comprising an amino acid sequence having at least 90%, at least 95%, at least 98%, at least 99%, or 100%, amino acid sequence identity to the amino acid sequence: DIQMTQSPSSLSASVGDRVTITCKASQDVSIGVAWYQQKPGKAPKLLIYSASYRYTGVPSRFSGS GSGTDFTLTISSLQPEDFATYYCQQYYIYPYTFGQGTKVEIK (SEQ ID NO:39); and b) a VH comprising an amino acid sequence having at least 90%, at least 95%, at least 98%, at least 99%, or 100%, amino acid sequence identity to the amino acid sequence:

(SEQ ID NO: 40)
EVQLVESGGGLVQPGGSLRLSCAASGFTFTDYTMDWVRQAPGKGLEWVA
DVNPNSGGSIYNQRFKGRFTLSVDRSKNTLYLQMNSLRAEDTAVYYCAR
NLGPSFYFDYWGQGTLVTVSS.

In some cases, an anti-Her2 antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-HER2 antibody can comprise a VL CDR1 having the amino acid sequence KASQDVSIGVA (SEQ ID NO:41); a VL CDR2 having the amino acid sequence SASYRY (SEQ ID NO:42); a VL CDR3 having the amino acid sequence QQYYIYPY (SEQ ID NO:43); a VH CDR1 having the amino acid sequence GFTFTDYTMD (SEQ ID NO:44); a VH CDR2 having the amino acid sequence ADVNPNSGGSIYNQRFKG (SEQ ID NO:45); and a VH CDR3 having the amino acid sequence ARNLGPSFYFDY (SEQ ID NO:46).

In some cases, an anti-Her2 antibody is a scFv. For example, in some cases, an anti-Her2 scFv comprises an amino acid sequence having at least 90%, at least 95%, at least 98%, at least 99%, or 100%, amino acid sequence identity to the following amino acid sequence:

(SEQ ID NO: 36)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSGGGGSGGGGSGGGGSDIQMTQSPSSLS
ASVGDRVTITCRASQDVNTAVAWYQQKPGKAPKLLIYSASFLYSGVPSR
FSGSRSGTDFTLTISSLQPEDFATYYCQQHYTTPPTFGQGTKVEIK.

### 2) Anti-CD19

Anti-CD19 antibodies are known in the art; and the VH and VL, or the VH and VL CDRs, of any anti-CD19 antibody can be included in a CAR. See e.g., WO 2005/012493.

In some cases, an anti-CD19 antibody includes a VL CDR1 comprising the amino acid sequence KASQSVDYDGDSYLN (SEQ ID NO:47); a VL CDR2 comprising the amino acid sequence DASNLVS (SEQ ID NO:48); and a VL CDR3 comprising the amino acid sequence QQSTEDPWT (SEQ ID NO:49). In some cases, an anti-CD19 antibody includes a VH CDR1 comprising the amino acid sequence SYWMN (SEQ ID NO:50); a VH CDR2 comprising the amino acid sequence QIWPGDGDTNYNGKFKG (SEQ ID NO:51); and a VH CDR3 comprising the amino acid sequence RETTTVGRYYYAMDY (SEQ ID NO:52). In some cases, an anti-CD19 antibody includes a VL CDR1 comprising the amino acid sequence KASQSVDYDGDSYLN (SEQ ID NO:47); a VL CDR2 comprising the amino acid sequence DASNLVS (SEQ ID NO:48); a VL CDR3 comprising the amino acid sequence QQSTEDPWT (SEQ ID NO:49); a VH CDR1 comprising the amino acid sequence SYWMN (SEQ ID NO:50); a VH CDR2 comprising the amino acid sequence QIWPGDGDTNYNGKFKG (SEQ ID NO:51); and a VH CDR3 comprising the amino acid sequence RETTTVGRYYYAMDY (SEQ ID NO:52).

In some cases, an anti-CD19 antibody is a scFv. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 53)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGGGGSGGGGSGGGGSQVQLQQSGAELVRPGSSVKIS
CKASGYAFSSYWMNWVKQRPGQGLEWIGQIWPGDGDTNYNGKFKGKATL
TADESSSTAYMQLSSLASEDSAVYFCARRETTTVGRYYYAMDYWGQGTT
VTVS.

### 3) Anti-Mesothelin

Anti-mesothelin antibodies are known in the art; and the VH and VL, or the VH and VL CDRs, of any anti-mesothelin antibody can be included in a CAR. See, e.g., U.S. 2019/0000944; WO 2009/045957; WO 2014/031476; U.S. Pat. No. 8,460,660; US 2013/0066055; and WO 2009/068204.

In some cases, an anti-mesothelin antibody comprises: a) a light chain comprising an amino acid sequence having at least 90%, at least 95%, at least 98%, at least 99%, or 100%, amino acid sequence identity to the following amino acid sequence:

DIALTQPASVSGSPGQSITISCTGTSSDIGGYNSVSWYQQHPGKAPKLMIYGVNNR PSGVSNRFSGSKSGNTASLTISGLQAEDEADYYCSSYDIESATPVFGGGTKLTVLGQPKAAPSVT LFPPSSEELQANKATLVCLISDFYPGAVTVAWKGDSSPVKAGVETTTPSKQSNNKYAASSYLSL TPEQWKSHRSYSCQVTHEGSTVEKTVAPTESS (SEQ ID NO:54); and
b) a heavy chain comprising an amino acid sequence having at least 90%, at least 95%, at least 98%, at least 99%, or 100%, amino acid sequence identity to the following amino acid sequence:

(SEQ ID NO: 55)
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some cases, an anti-mesothelin antibody comprises a VL present in the light chain amino acid sequence provided above; and a VH present in the heavy chain amino acid sequence provided above. For example, an anti-mesothelin antibody can comprise: a) a VL comprising an amino acid sequence having at least 90%, at least 95%, at least 98%, at least 99%, or 100%, amino acid sequence identity to the amino acid sequence: DIALTQPASVSGSPGQSITISCTGTSSDIGGYNSVSWYQQHPGKAPKLMIYGVNNRPSGVSNRFS GSKSGNTASLTISGLQAEDEADYYCSSYDIESATPVFGGGTK (SEQ ID NO:56); and b) a VH comprising an amino acid sequence having at least 90%, at least 95%, at least 98%, at least 99%, or 100%, amino acid sequence identity to the amino acid sequence:

(SEQ ID NO: 57)
QVELVQSGAEVKKPGESLKISCKGSGYSFTSYWIGWVRQAPGKGLEWMG
IIDPGDSRTRYSPSFQGQVTISADKSISTAYLQWSSLKASDTAMYYCAR
GQLYGGTYMDGWGQGTLVTVSS.

In some cases, an anti-mesothelin antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-mesothelin antibody can comprise a VL CDR1 having the amino acid sequence TGTSSDIGGYNSVS (SEQ ID NO:58); a VL CDR2 having the amino acid sequence LMIYGVNNRPS (SEQ ID NO:59); a VL CDR3 having the amino acid sequence SSYDIESATP (SEQ ID NO:60); a VH CDR1 having the amino acid sequence GYSFTSYWIG (SEQ ID NO:61); a VH CDR2 having the amino acid sequence WMGIIDPGDSRTRYSP (SEQ ID NO:62); and a VH CDR3 having the amino acid sequence GQLYGGTYMDG (SEQ ID NO:63).

An anti-mesothelin antibody can be a scFv. As one non-limiting example, an anti-mesothelin scFv can comprise the following amino acid sequence:

(SEQ ID NO: 4)
QVQLQQSGAEVKKPGASVKVSCKASGYTFTGYYMHWVRQAPGQGLEWMG
RINPNSGGTNYAQKFQGRVTMTRDTSISTAYMELSRLRSEDTAVYYCAR
GRYYGMDVWGQGTMVTVSSGGGGSGGGGSGGGGSGGGGSEIVLTQSPAT
LSLSPGERATISCRASQSVSSNFAWYQQRPGQAPRLLIYDASNRATGIP
PRFSGSGSGTDFTLTISSLEPEDFAAYYCHQRSNWLYTFGQGTKVDIK,

where VH and CDR3 are underlined; and VL CDR1, CDR2, and CDR3 are bolded and underlined.

As one non-limiting example, an anti-mesothelin scFv can comprise the following amino acid sequence:

(SEQ ID NO: 65)
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
DLRRTVVTPRAYYGMDVWGQGTTVTVSSGGGGSGGGGSGGGGSGGGGSD
IQLTQSPSTLSASVGDRVTITCQASQDISNSLNWYQQKAGKAPKLLIYD
ASTLETGVPSRFSGSGSGTDFSFTISSLQPEDIATYYCQQHDNLPLTFG
QGTKVEIK,

where VH CDR1, CDR2, and CDR3 are underlined; and VL CDR1, CDR2, and CDR3 are bolded and underlined.

### 4) Anti-BCMA

Anti-BCMA (B-cell maturation antigen) antibodies are known in the art; and the VH and VL, or the VH and VL CDRs, of any anti-BCMA antibody can be included in a CAR. See, e.g., WO 2014/089335; US 2019/0153061; and WO 2017/093942.

In some cases, an anti-BCMA antibody comprises: a) a light chain comprising an amino acid sequence having at least 90%, at least 95%, at least 98%, at least 99%, or 100%, amino acid sequence identity to the following amino acid sequence:

QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLGQPKAAPSV TLFPPSSEELQANKATLVCLISDFYPGAVTVAWKADSSPVKAGVETTTPDSKQSNNKYAASSYL SLTPEQWKSHRSYSCQVTHEGSTVEKTVAPTECS (SEQ ID NO:66); and
b) a heavy chain comprising an amino acid sequence having at least 90%, at least 95%, at least 98%, at least 99%, or 100%, amino acid sequence identity to the following amino acid sequence:

(SEQ ID NO: 67)
EVQLVESGGGLVKPGGSLRLSCAASGFTFGDYALSWFRQAPGKGLEWVG
VSRSKAYGGTTDYAASVKGRFTISRDDSKSTAYLQMNSLKTEDTAVYYC
ASSGYSSGWTPFDYWGQGTLVTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNH
YTQKSLSLSPGK.

In some cases, an anti-BCMA antibody comprises a VL present in the light chain amino acid sequence provided above; and a VH present in the heavy chain amino acid sequence provided above. For example, an anti-BCMA antibody can comprise: a) a VL comprising an amino acid sequence having at least 90%, at least 95%, at least 98%, at least 99%, or 100%, amino acid sequence identity to the amino acid sequence:

QSVLTQPPSASGTPGQRVTISCSGSSSNIGSNTVNWYQQLPGTAPKLLIFNYHQRP SGVPDRFSGSKSGSSASLAISGLQSEDEADYYCAAWDDSLNGWVFGGGTKLTVLG (SEQ ID NO:68); and b) a VH comprising an amino acid sequence having at least 90%, at least 95%, at least 98%, at least 99%, or 100%, amino acid sequence identity to the amino acid sequence:

(SEQ ID NO: 69)
EVQLVESGGGLVKPGGSLRLSCAASGFTFGDYALSWFRQAPGKGLEWVG
VSRSKAYGGTTDYAASVKGRFTISRDDSKSTAYLQMNSLKTEDTAVYYC
ASSGYSSGWTPFDYWGQGTLVTVSSASTKGPSV.

In some cases, an anti-BCMA antibody comprises VL CDR1, VL CDR2, and VL CDR3 present in the light chain amino acid sequence provided above; and VH CDR1, CDR2, and CDR3 present in the heavy chain amino acid sequence provided above. In some cases, the V_{H} and V_{L} CDRs are as defined by Kabat (see, e.g., Table 1, above; and Kabat 1991). In some cases, the V_{H} and V_{L} CDRs are as defined by Chothia (see, e.g., Table 1, above; and Chothia 1987).

For example, an anti-BCMA antibody can comprise a VL CDR1 having the amino acid sequence SSNIGSNT (SEQ ID NO:70), a VL CDR2 having the amino acid sequence NYH, a VL CDR3 having the amino acid sequence AAWDDSLNGWV (SEQ ID NO:71)), a VH CDR1 having the amino acid sequence GFTFGDYA (SEQ ID NO:72), a VH CDR2 having the amino acid sequence SRSKAYGGTT (SEQ ID NO:73), and a VH CDR3 having the amino acid sequence ASSGYSSGWTPFDY (SEQ ID NO:74).

An anti-BCMA antibody can be a scFv. As one non-limiting example, an anti-BCMA scFv can comprise the following amino acid sequence:

(SEQ ID NO: 75)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYNGYDVLDNWGQGTLVTVSSGGGGSGGGGSGGGGSGGGGSDIQMTQ
SPSSLSASVGDRVTITCSASQDISNYLNWYQQKPGKAPKLLIYYTSNLH
SGVPSRFSGSGSGTDFTLTISSLQPEDFATYYCQQYRKLPWTFGQGTKL
EIKR.

As another example, an anti-BCMA scFv can comprise the following amino acid sequence:

(SEQ ID NO: 76)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRGGGGSGGGGSGGGGSGGGGSQVQLVQSGAEVKKPGSSVK
VSCKASGGTFSNYWMHWVRQAPGQGLEWMGATYRGHSDTYYNQKFKGRV
TITADKSTSTAYMELSSLRSEDTAVYYCARGAIYNGYDVLDNWGQGTLV
TVSS.

In some cases, an anti-BCMA antibody can comprise a VL CDR1 having the amino acid sequence SASQDISNYLN (SEQ ID NO:77); a VL CDR2 having the amino acid sequence YTSNLHS (SEQ ID NO:78); a VL CDR3 having the amino acid sequence QQYRKLPWT (SEQ ID NO:79); a VH CDR1 having the amino acid sequence NYWMH (SEQ ID NO:80); a VH CDR2 having the amino acid sequence ATYRGHSDTYYNQKFKG (SEQ ID NO:81); and a VH CDR3 having the amino acid sequence GAIYNGYDVLDN (SEQ ID NO:82).

In some cases, an anti-BCMA antibody comprises: a) a light chain comprising an amino acid sequence having at least 90%, at least 95%, at least 98%, at least 99%, or 100%, amino acid sequence identity to the following amino acid sequence:

(SEQ ID NO: 83)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKR.

In some cases, an anti-BCMA antibody comprises: a) a heavy chain comprising an amino acid sequence having at least 90%, at least 95%, at least 98%, at least 99%, or 100%, amino acid sequence identity to the following amino acid sequence:

(SEQ ID NO: 84)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

In some cases, an anti-BCMA antibody (e.g., an antibody referred to in the literature as belantamab) comprises a light chain comprising the amino acid sequence: DIQMTQSPSSLSASVGDRVTITCSASQDISNYLNWYQQKPGKAPKLLIYYTSNLHSGVPSRFSGS GSGTDFTLTISSLQPEDFATYYCQQYRKLPWTFGQGTKLEIKR (SEQ ID NO:83); and a heavy chain comprising the amino acid sequence:

(SEQ ID NO: 84)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYDGYDVLDNWGQGTLVTVSS.

In some cases, the anti-BCMA antibody has a cancer chemotherapeutic agent linked to the antibody. For example, in some cases, the anti-BCMA antibody is GSK2857916 (belantamab-mafodotin), where monomethyl auristatin F (MMAF) is linked via a maleimidocaproyl linker to the anti-BCMA antibody belantamab.

### 5) Anti-MUC1

In some cases, an antigen-binding polypeptide present in a CAR is a single-chain Fv specific for MUC1. See, e.g., Singh et al. (2007) Mol. Cancer Ther. 6:562; Thie et al. (2011) PLoSOne 6:e15921; Imai et al. (2004) Leukemia 18:676; Posey et al. (2016) Immunity 44:1444; EP3130607; EP3164418; WO 2002/044217; and US 2018/0112007. In some cases, an antigen-binding polypeptide present in a CAR is a scFv specific for the MUC1 peptide VTSAPDTRPAPGSTAPPAHG (SEQ ID NO:85). In some cases, a TTP is a scFv specific for the MUC1 peptide SNIKFRPGSVVVQLTLAFREGTINVHDVETQFNQYKTEAASRY (SEQ ID NO:86). In some cases, an antigen-binding polypeptide present in a CAR is a scFv specific for the MUC1 peptide SVVVQLTLAFREGTINVHDVETQFNQYKTEAASRY (SEQ ID NO:87). In some cases, a TTP is a scFv specific for the MUC1 peptide LAFREGTINVHDVETQFNQY (SEQ ID NO:88). In some cases, an antigen-binding polypeptide present in a CAR is a scFv specific for the MUC1 peptide SNIKFRPGSVVVQLTLAAFREGTIN (SEQ ID NO:89).

As an example, an anti-MUC1 antibody can comprise: a VH CDR1 having the amino acid sequence RYGMS (SEQ ID NO:90); a VH CDR2 having the amino acid sequence TISGGGTYIYYPDSVKG (SEQ ID NO:91); a VH CDR3 having the amino acid sequence DNYGRNYDYGMDY (SEQ ID NO:92); a VL CDR1 having the amino acid sequence SATSSVSYIH (SEQ ID NO:93); a VL CDR2 having the amino acid sequence STSNLAS (SEQ ID NO:94); and a VL CDR3 having the amino acid sequence QQRSSSPFT (SEQ ID NO:95). See, e.g., US 2018/0112007.

As another example, an anti-MUC1 antibody can comprise a VH CDR1 having the amino acid sequence GYAMS (SEQ ID NO:96); a VH CDR2 having the amino acid sequence TISSGGTYIYYPDSVKG (SEQ ID NO:97); a VH CDR3 having the amino acid sequence LGGDNYYEYFDV (SEQ ID NO:98); a VL CDR1 having the amino acid sequence RASKSVSTSGYSYMH (SEQ ID NO:99); a VL CDR2 having the amino acid sequence LASNLES (SEQ ID NO:100); and a VL CDR3 having the amino acid sequence QHSRELPFT (SEQ ID NO:101). See, e.g., US 2018/0112007.

As another example, an anti-MUC1 antibody can comprise a VH CDR1 having the amino acid sequence DYAMN (SEQ ID NO:102); a VH CDR2 having the amino acid sequence VISTFSGNINFNQKFKG (SEQ ID NO:103); a VH CDR3 having the amino acid sequence SDYYGPYFDY (SEQ ID NO:104); a VL CDR1 having the amino acid sequence RSSQTIVHSNGNTYLE (SEQ ID NO:105); a VL CDR2 having the amino acid sequence KVSNRFS (SEQ ID NO:106); and a VL CDR3 having the amino acid sequence (FQGSHVPFT (SEQ ID NO:107). See, e.g., US 2018/0112007.

As another example, an anti-MUC1 antibody can comprise a VH CDR1 having the amino acid sequence GYAMS (SEQ ID NO:96); a VH CDR2 having the amino acid sequence TISSGGTYIYYPDSVKG (SEQ ID NO:97); a VH CDR3 having the amino acid sequence LGGDNYYEY (SEQ ID NO:108); a VL CDR1 having the amino acid sequence TASKSVSTSGYSYMH (SEQ ID NO:109); a VL CDR2 having the amino acid sequence LVSNLES (SEQ ID NO:110); and a VL CDR3 having the amino acid sequence QHIRELTRSE (SEQ ID NO:111). See, e.g., US 2018/0112007.

### 6) Anti-MUC16

In some cases, an antigen-binding polypeptide present in a CAR is specific for a MUC16 polypeptide present on a cancer cell. See, e.g., US 2018/0118848; and US 2018/0112008. In some cases, a MUC16-specific antigen-binding polypeptide is a scFv. In some cases, a MUC16-specific antigen-binding polypeptide is a nanobody.

As one example, an anti-MUC16 antibody can comprise a VH CDR1 having the amino acid sequence GFTFSNYY (SEQ ID NO: 112); a VH CDR2 having the amino acid sequence ISGRGSTI (SEQ ID NO:113); a VH CDR3 having the amino acid sequence VKDRGGYSPY (SEQ ID NO:114); a VL CDR1 having the amino acid sequence QSISTY (SEQ ID NO:115); a VL CDR2 having the amino acid sequence TAS; and a VL CDR3 having the amino acid sequence QQSYSTPPIT (SEQ ID NO: 116). See, e.g., US 2018/0118848.

### 7) Examples of Antigen-Binding Domains

In some cases, a suitable CAR comprises a scFv specific for CD19. For example, in some cases, an anti-CD19 scFv comprises an amino acid sequence having at least 90%, at least 95%, at least 98%, at least 99%, or 100%, amino acid sequence identity to the following amino acid sequence:

(SEQ ID NO: 53)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIKGGGGSGGGGSGGGGSQVQLQQSGAELVRPGSSVKIS
CKASGYAFSSYWMNWVKQRPGQGLEWIGQIWPGDGDTNYNGKFKGKATL
TADESSSTAYMQLSSLASEDSAVYFCARRETTTVGRYYYAMDYWGQGTT
VTVS.

In some cases, a suitable CAR comprises a scFv specific for mesothelin. For example, in some cases, an anti-mesothelin scFv comprises an amino acid sequence having at least 90%, at least 95%, at least 98%, at least 99%, or 100%, amino acid sequence identity to the following amino acid sequence:

(SEQ ID NO: 64)
QVQLQQSGAEVKKPGASVKVSCKASGYTFTGYYMHWVRQAPGQGLEWMG
RINPNSGGTNYAQKFQGRVTMTRDTSISTAYMELSRLRSEDTAVYYCAR
GRYYGMDVWGQGTMVTVSSGGGGSGGGGSGGGGSGGGGSEIVLTQSPAT
LSLSPGERATISCRASQSVSSNFAWYQQRPGQAPRLLIYDASNRATGIP
PRFSGSGSGTDFTLTISSLEPEDFAAYYCHQRSNWLYTFGQGTKVDIK.

In some cases, an anti-mesothelin scFv comprises an amino acid sequence having at least 90%, at least 95%, at least 98%, at least 99%, or 100%, amino acid sequence identity to the following amino acid sequence:

(SEQ ID NO: 65)
QVQLVQSGAEVKKPGASVKVSCKASGYTFTGYYMHWVRQAPGQGLEWMG
WINPNSGGTNYAQKFQGRVTMTRDTSISTAYMELSRLRSDDTAVYYCAR
DLRRTVVTPRAYYGMDVWGQGTTVTVSSGGGGSGGGGSGGGGSGGGGSD
IQLTQSPSTLSASVGDRVTITCQASQDISNSLNWYQQKAGKAPKLLIYD
ASTLETGVPSRFSGSGSGTDFSFTISSLQPEDIATYYCQQHDNLPLTFG
QGTKVEIK.

In some cases, a suitable CAR comprises a scFv specific for B-cell maturation antigen (BCMA). For example, in some cases, an anti-BCMA scFv comprises an amino acid sequence having at least 90%, at least 95%, at least 98%, at least 99%, or 100%, amino acid sequence identity to the following amino acid sequence:

(SEQ ID NO: 75)
QVQLVQSGAEVKKPGSSVKVSCKASGGTFSNYWMHWVRQAPGQGLEWMG
ATYRGHSDTYYNQKFKGRVTITADKSTSTAYMELSSLRSEDTAVYYCAR
GAIYNGYDVLDNWGQGTLVTVSSGGGGSGGGGSGGGGSGGGGSDIQMTQ
SPSSLSASVGDRVTITCSASQDISNYLNWYQQKPGKAPKLLIYYTSNLH
SGVPSRFSGSGSGTDFTLTISSLQPEDFATYYCQQYRKLPWTFGQGTKL
EIKR.

In some cases, an anti-BCMA scFv comprises an amino acid sequence having at least 90%, at least 95%, at least 98%, at least 99%, or 100%, amino acid sequence identity to the following amino acid sequence:

(SEQ ID NO: 76)
DIQMTQSPSSLSASVGDRVTITCSASQDISNYLNWYQQKPGKAPKLLIY
YTSNLHSGVPSRFSGSGSGTDFTLTISSLQPEDFATYYCQQYRKLPWTF
GQGTKLEIKRGGGGSGGGGSGGGGSGGGGSQVQLVQSGAEVKKPGSSVK
VSCKASGGTFSNYWMHWVRQAPGQGLEWMGATYRGHSDTYYNQKFKGRV
TITADKSTSTAYMELSSLRSEDTAVYYCARGAIYNGYDVLDNWGQGTLV
TVSS.

### Hinge Region

As noted above, a CAR can include a hinge region between the extracellular domain and the transmembrane domain. As used herein, the term “hinge region” refers to a flexible polypeptide connector region (also referred to herein as “hinge” or “spacer”) providing structural flexibility and spacing to flanking polypeptide regions and can consist of natural or synthetic polypeptides. The hinge region can include complete hinge region derived from an antibody of a different class or subclass from that of the CH1 domain. The term “hinge region” can also include regions derived from CD8 and other receptors that provide a similar function in providing flexibility and spacing to flanking regions.

The hinge region can have a length of from about 4 amino acids to about 50 amino acids, e.g., from about 4 aa to about 10 aa, from about 10 aa to about 15 aa, from about 15 aa to about 20 aa, from about 20 aa to about 25 aa, from about 25 aa to about 30 aa, from about 30 aa to about 40 aa, or from about 40 aa to about 50 aa.

As non-limiting examples, an immunoglobulin hinge region can include one of the following amino acid sequences: DKTHT (SEQ ID NO:117); CPPC (SEQ ID NO:118); CPEPKSCDTPPPCPR (SEQ ID NO:119); ELKTPLGDTTHT (SEQ ID NO:120); KSCDKTHTCP (SEQ ID NO:121); KCCVDCP (SEQ ID NO:122); KYGPPCP (SEQ ID NO:123); EPKSCDKTHTCPPCP (SEQ ID NO:124) (human IgG1 hinge); ERKCCVECPPCP (SEQ ID NO:125) (human IgG2 hinge); ELKTPLGDTTHTCPRCP (SEQ ID NO:126) (human IgG3 hinge); SPNMVPHAHHAQ (SEQ ID NO:127) (human IgG4 hinge); and the like. The hinge region can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:128), or a variant thereof.

### Transmembrane Domain

Any transmembrane (TM) domain that provides for insertion of a polypeptide into the cell membrane of a eukaryotic (e.g., mammalian) cell is suitable for use. The transmembrane region of a CAR can be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R .alpha., ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. The transmembrane domain can be synthetic, in which case it can comprise predominantly hydrophobic residues such as leucine and valine. In some cases, a triplet of phenylalanine, tryptophan and valine will be found at each end of a synthetic transmembrane domain.

As one non-limiting example, the TM sequence IYIWAPLAGTCGVLLLSLVITLYC (SEQ ID NO:129) can be used. Additional non-limiting examples of suitable TM sequences include: a) CD8 beta derived TM: LGLLVAGVLVLLVSLGVAIHLCC (SEQ ID NO:130); b) CD4 derived TM: ALIVLGGVAGLLLFIGLGIFFCVRC (SEQ ID NO:131); c) CD3 zeta derived TM: LCYLLDGILFIYGVILTALFLRV (SEQ ID NO:132); d) CD28 derived TM: WVLVVVGGVLACYSLLVTVAFIIFWV (SEQ ID NO:133); e) CD134 (OX40) derived TM: VAAILGLGLVLGLLGPLAILLALYLL (SEQ ID NO:134); and f) CD7 derived TM: ALPAALAVISFLLGLGLGVACVLA (SEQ ID NO:135).

### Intracellular Domain—Co-Stimulatory Polypeptide

The intracellular portion (cytoplasmic domain) of a CAR can comprise one or more co-stimulatory polypeptides. Non-limiting examples of suitable co-stimulatory polypeptides include, but are not limited to, 4-1BB (CD137), CD28, ICOS, OX-40, BTLA, CD27, CD30, GITR, and HVEM. Suitable co-stimulatory polypeptides include, e.g.: 1) a 4-1BB polypeptide having at least 90%, at least 95%, at least 98%, or 100%, amino acid sequence identity to the following amino acid sequence: KRGRKKLLYIFKQPFMRPVQTTQEEDGCSCRFPEEEEGGCEL (SEQ ID NO:136); 2) a CD28 polypeptide having at least 90%, at least 95%, at least 98%, or 100%, amino acid sequence identity to the following amino acid sequence: FWVRSKRSRLLHSDYMNMTPRRPGPTRKHYQPYAPPRDFAAYRS (SEQ ID NO:137); 3) an ICOS polypeptide having at least 90%, at least 95%, at least 98%, or 100%, amino acid sequence identity to the following amino acid sequence: TKKKYSSSVHDPNGEYMFMRAVNTAKKSRLTDVTL (SEQ ID NO:138); 4) an OX40 polypeptide having at least 90%, at least 95%, at least 98%, or 100%, amino acid sequence identity to the following amino acid sequence: RRDQRLPPDAHKPPGGGSFRTPIQEEQADAHSTLAKI (SEQ ID NO:139); 5) a BTLA polypeptide having at least 90%, at least 95%, at least 98%, or 100%, amino acid sequence identity to the following amino acid sequence: CCLRRHQGKQNELSDTAGREINLVDAHLKSEQTEASTRQNSQVLLSETGIYDNDPDLCFRMQEG SEVYSNPCLEENKPGIVYASLNHSVIGPNSRLARNVKEAPTEYASICVRS (SEQ ID NO:140); 6) a CD27 polypeptide having at least 90%, at least 95%, at least 98%, or 100%, amino acid sequence identity to the following amino acid sequence: HQRRKYRSNKGESPVEPAEPCRYSCPREEEGSTIPIQEDYRKPEPACSP (SEQ ID NO:141); 7) a CD30 polypeptide having at least 90%, at least 95%, at least 98%, or 100%, amino acid sequence identity to the following amino acid sequence: RRACRKRIRQKLHLCYPVQTSQPKLELVDSRPRRSSTQLRSGASVTEPVAEERGLMSQPLMETC HSVGAA YLESLPLQDASPAGGPSSPRDLPEPRV STEHTNNKIEK1YIMKADTVIVGTVKAELPEG RGLAGPAEPELEEELEADHTPHYPEQETEPPLGSCSDVMLSVEEEGKEDPLPTAASGK (SEQ ID NO:142); 8) a GITR polypeptide having at least 90%, at least 95%, at least 98%, or 100%, amino acid sequence identity to the following amino acid sequence: HIWQLRSQCMWPRETQLLLEVPPSTEDARSCQFPEEERGERSAEEKGRLGDLWV (SEQ ID N0143/); and 9) an HVEM polypeptide having at least 90%, at least 95%, at least 98%, or 100%, amino acid sequence identity to the following amino acid sequence: CVKRRKPRGDVVKViVSVQRKRQEAEGEATViEALQAPPDVTTVAVEETiPSFTGRSPNH (SEQ ID NO:144). The co-stimulatory polypeptide can have a length of from about 30 aa to about 35 aa, from about 35 aa to about 40 aa, from about 40 aa to about 45 aa, from about 45 aa to about 50 aa, from about 50 aa to about 55 aa, from about 55 aa to about 60 aa, from about 60 aa to about 65 aa, or from about 65 aa to about 70 aa.

### Intracellular Domain—Signaling Polypeptide

The intracellular portion of a CAR can comprise a signaling polypeptide. Suitable signaling polypeptides include, e.g., an immunoreceptor tyrosine-based activation motif (ITAM)-containing intracellular signaling polypeptide. An ITAM motif is YX₁X₂L/I, where X₁ and X₂ are independently any amino acid. In some cases, the intracellular signaling domain of a subject CAR comprises 1, 2, 3, 4, or 5 ITAM motifs. In some cases, an ITAM motif is repeated twice in an intracellular signaling domain, where the first and second instances of the ITAM motif are separated from one another by 6 to 8 amino acids, e.g., (YX₁X₂L/I)(X₃)ₙ(YX₁X₂L/I), where n is an integer from 6 to 8, and each of the 6-8 X₃ can be any amino acid. In some cases, the intracellular signaling domain of a CAR comprises 3 ITAM motifs.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12; FCER1G (Fc epsilon receptor 1 gamma chain); CD3D (CD3 delta); CD3E (CD3 epsilon); CD3G (CD3 gamma); CD3Z (CD3 zeta); and CD79A (antigen receptor complex-associated protein alpha chain).

### TCR

As noted above, a mCTL of the present disclosure comprises (e.g., expresses on its surface) a TCR specific for the, HPV E7₁₁-19/20 TCR, i.e., a TCR specific for HPV E7 peptide YMLDLQPETT (SEQ ID NO:1) or YMLDLQPET (SEQ ID NO:2) in a complex with class I MHC polypeptides (an MHC class I heavy chain polypeptide and a P2-microglobulin (p2M) polypeptide). The TCR is heterologous to the mCTL; i.e., the TCR is one that does not naturally occur in the mCTL.

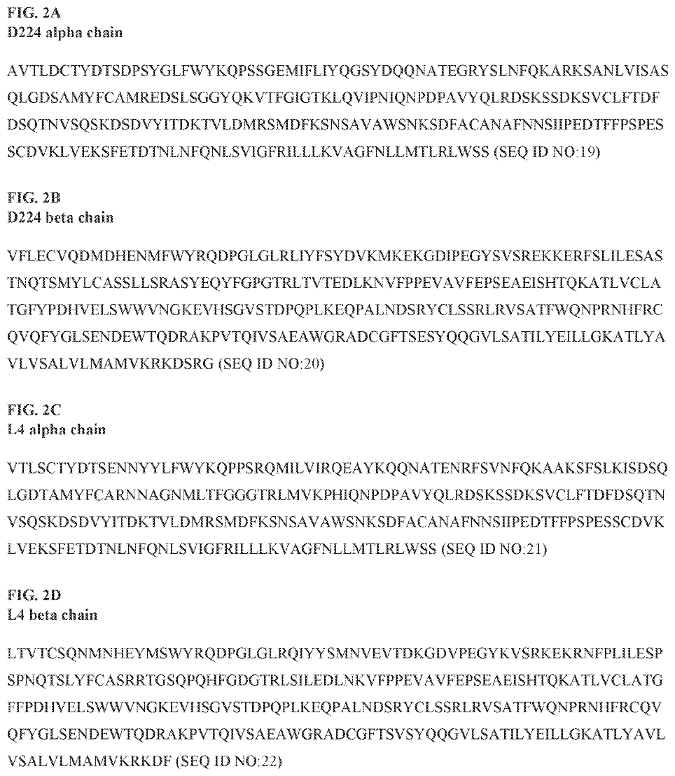

In some cases, a mCTL comprises a heterologous TCR comprising: (i) an alpha chain comprising an amino acid sequence having at least 90%, at least 95%, at least 98%, at least 99%, or 100%, amino acid sequence identity to the D224 alpha chain amino acid sequence depicted in FIG. 2A; and (ii) a beta comprising an amino acid sequence having at least 90%, at least 95%, at least 98%, at least 99%, or 100%, amino acid sequence identity to the D224 alpha chain amino acid sequence depicted in FIG. 2B. In some cases, a mCTL comprises a heterologous TCR comprising (i) an alpha chain having the amino acid sequence depicted in FIG. 2A, and (ii) a beta chain having the amino acid sequence depicted in FIG. 2B.

In some cases, a mCTL comprises a heterologous TCR comprising: (i) an alpha chain comprising an amino acid sequence having at least 90%, at least 95%, at least 98%, at least 99%, or 100%, amino acid sequence identity to the D224 alpha chain amino acid sequence depicted in FIG. 2C; and (ii) a beta comprising an amino acid sequence having at least 90%, at least 95%, at least 98%, at least 99%, or 100%, amino acid sequence identity to the D224 alpha chain amino acid sequence depicted in FIG. 2D. In some cases, a mCTL comprises a heterologous TCR comprising (i) an alpha chain having the amino acid sequence depicted in FIG. 2C, and (ii) a beta chain having the amino acid sequence depicted in FIG. 2D.

### Compositions Comprising MCTLS

The present disclosure provides a composition comprising mCTLs of the present disclosure. The present disclosure provides a composition comprising T cells, where the percentage of T cells that are mCTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

A composition of the present disclosure can comprise, in addition to mCTLs of the present disclosure, one or more of: a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like. A composition comprising mCTLs can include a liquid culture medium that provides for maintenance of the cells.

The composition may comprise one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

A pharmaceutical composition can comprise a quantity of mCTLs of the present disclosure, and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins, or where such detectable pyrogens and/or other toxins are present at a level within acceptable limits set by an applicable regulatory agency, e.g., the U.S. Food and Drug Administration (USF&DA).

The present disclosure provides a container comprising mCTLs of the present disclosure, e.g., a liquid composition. The container can be, e.g., a syringe, an ampoule, and the like. In some cases, the container is sterile. In some cases, both the container and the composition are sterile.

In some cases, mCTLs are present in a liquid composition. Thus, the present disclosure provides compositions (e.g., liquid compositions, including pharmaceutical compositions) comprising mCTLs. In some cases, a composition of the present disclosure comprises: a) mCTLs; and b) saline (e.g., 0.9% NaCl). In some cases, the composition is sterile. In some cases, the composition is suitable for administration to a human subject, e.g., where the composition is sterile and is free of detectable pyrogens and/or other toxins, or where such detectable pyrogens and/or other toxins are present at a level within acceptable limits set by an applicable regulatory agency, e.g., the USF&DA. Thus, the present disclosure provides a composition comprising: a) mCTLs; and b) saline (e.g., 0.9% NaCl), where the composition is sterile and is free of detectable pyrogens and/or other toxins, or where such detectable pyrogens and/or other toxins are present at a level within acceptable limits set by an applicable regulatory agency, e.g., the USF&DA. In some cases, a composition further comprises a TMP as described herein.

The present disclosure provides an admixture of mCTLs of the present disclosure and a TMP comprising an HPV16 peptide comprising the amino acid sequence YMLDLQPETT (SEQ ID NO:1). The present disclosure provides an admixture of mCTLs of the present disclosure and a TMP comprising an HPV16 peptide comprising the amino acid sequence YMLDLQPET (SEQ ID NO:2).

### T-Cell Modulatory Polypeptide (TMP)

A T-cell modulatory polypeptide (TMP) suitable for use herein includes either a single-chain polypeptide or a heterodimeric polypeptide that comprises: (i) a peptide, e.g., an HPV16 peptide comprising the amino acid sequence YMLDLQPETT (SEQ ID NO:1) or YMLDLQPET (SEQ ID NO:2); (ii) a p2 microglobulin (p2M) polypeptide; (iii) an immunomodulatory polypeptide (or “MOD”) such as an IL-2 polypeptide; (iv) a major histocompatibility complex class I (MHC class I) heavy chain polypeptide; and (v) a scaffold polypeptide such as an immunoglobulin (Ig) Fc polypeptide.

For example, a TMP can comprise a heterodimeric polypeptide comprising: a) a) a first polypeptide comprising: (i) an HPV16 peptide comprising the amino acid sequence YMLDLQPETT (SEQ ID NO:1); (ii) a linker; and (iii) a p2 microglobulin (p2M) polypeptide; and b) a second polypeptide comprising: (i) two copies of an IL-2 polypeptide; (ii) a major histocompatibility complex class I (MHC class I) heavy chain polypeptide; and (iii) an immunoglobulin (Ig) Fc polypeptide, e.g., a variant IgG1 Fc polypeptide that has a substantially reduced ability to effect complement-dependent cytotoxicity (CDC) or antibody-dependent cell cytotoxicity (ADCC).

The β2M polypeptide can comprise an amino acid sequence having at least 90%, at least 95%, at least 98%, at least 99%, or 100%, amino acid sequence identity to the following P2M amino acid sequence: IQRTPKIQVY SRHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:3). In some cases, a P2M polypeptide comprises an amino acid sequence having at least 90%, at least 95%, at least 98%, at least 99%, or 100%, amino acid sequence identity to the following p2M amino acid sequence: IQRTPKIQVY SCHPAENGKS NFLNCYVSGF HPSDIEVDLLKNGERIEKVE HSDLSFSKDW SFYLLYYTEF TPTEKDEYAC RVNHVTLSQP KLVKWDRDM (SEQ ID NO:4), where amino acid 12 is a Cys. In some cases, a f32M polypeptide comprises the amino acid sequence IQRTPKIQVY SCHPAENGKS NFLNCYVSGF HPSDIEVDLLKNGERIEKVE HSDLSFSKDW SFYLLYYTEF TPTEKDEYAC RVNHVTLSQP KIVKWDRDM (SEQ ID NO:4).

In some cases, the IL-2 polypeptide is a variant of a naturally occurring costimulatory protein, which variant exhibits a reduced affinity for its counterpart (cognate) costimulatory protein on the T cell (e.g., IL-2R) as compared to the affinity of the naturally occurring IL-2 polypeptide for the counterpart costimulatory protein (IL-2R). In some cases, an IL-2 variant exhibits decreased binding to IL-2Rα, thereby minimizing or substantially reducing the activation of Tregs by the IL-2 variant. Alternatively, or additionally, in some cases, an IL-2 variant MOD exhibits decreased binding to IL-2Rβ such that the IL-2 variant MOD exhibits an overall reduced affinity for IL-2R. In some cases, an IL-2 variant MOD exhibits both properties, i.e., it exhibits decreased or substantially no binding to IL-2Rα, and also exhibits decreased binding to IL-2Rβ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2. In some cases, an IL-2 variant MOD of this disclosure exhibits decreased binding to IL-2Ry. This decreased binding to IL-2Ry may be in addition to the decreased binding to IL-2Rα and/or IL-2Rβ.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:5), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLE EELKPLEEVL NLAQSKNFHL RPRDLISNIN VIVLELKGSE TTFMCEYADE TATIVEFLNRWITFCQSIIS TLT (SEQ ID NO:5). In some cases, a variant IL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNR WITFCQSIIS TLT (SEQ ID NO:6). In some cases, a TMP comprises two copies of such a variant IL-2 polypeptide joined by a linker.

As noted above, a TMP comprises an MHC class I heavy chain polypeptide. In some cases, the TMP comprises an MHC class I HLA-A allele heavy chain. In some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

(SEQ ID NO: 7)
GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRM
YGCDVGSDWRFLRGYHQYAYDGKDYTALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPAGDGTFQKWA
AVVVPSGQEQRYTCHVQHEGLPKPLTLRWE.

In some cases, the MHC class I heavy chain polypeptide comprises an Ala at position 84 and a Cys at position 236. Thus, e.g., in some cases, the MHC class I heavy chain polypeptide comprises an amino acid sequence having at least 80%, at least 85%, at least 90%, at least 95%, at least 98%, at least 99%, or 100%, amino acid sequence identity to the following HLA-A heavy chain amino acid sequence:

GSHSMRYFFTSVSRPGRGEPRFIAVGYVDDTQFVRFDSDAASQRMEPRAPWIEQ EGPEYWDGETRKVKAHSQTHRVDLGTLRGAYNQSEAGSHTVQRMYGCDVGSDWRFLRGYHQ YAYDGKDYIALKEDLRSWTAADMAAQTTKHKWEAAHVAEQLRAYLEGTCVEWLRRYLENG KETLQRTDAPKTHMTHHAVSDHEATLRCWALSFYPAEITLTWQRDGEDQTQDTELVETRPCGD GTFQKWAAVVVPSGQEQRYTCHVQHEGLPKPLTLRWE (SEQ ID NO:8), where amino acid 84 is an Ala and amino acid 236 is a Cys.

As noted above, a TMP comprises an Ig Fc polypeptide. An Ig Fc polypeptide is also referred to herein as an “Fc polypeptide.” The Ig Fc polypeptide of a TMP can be a human IgG1 Fc, a human IgG2 Fc, a human IgG3 Fc, a human IgG4 Fc, etc., or a variant of a wild-type Ig Fc polypeptide. Variants include naturally occurring variants, non-naturally occurring variants, and combinations thereof. For example, the Ig Fc can be a variant of a Fe polypeptide such as a human IgG1 Fc, which variant has a reduced or substantially eliminated ability to effect complement-dependent cytotoxicity (CDC) or antibody-dependent cell cytotoxicity (ADCC).

In some cases, the Fe polypeptide present in a TMP is an IgG1 Fe polypeptide, or a variant of an IgG1 Fe polypeptide. For example, in some cases, the Fe polypeptide present in a TMP comprises an amino acid sequence having at least 80%, at least 85%, at least 90%, at least 95%, at least 98%, at least 99%, or 100%, amino acid sequence identity to the following human IgG1 Fc polypeptide amino acid sequence: DKTHTCPPCP APELLGGPSV FLFPPKPKDT LMISRTPEVT CVVVDVSHED PEVKFNWYVD GVEVHNAKTK PREEQYNSTY RVVSVLTVLH QDWLNGKEYK CKVSNKALPA PIEKTISKAK GQPREPQVYT LPPSRDELTK NQVSLTCLVK GFYPSDIAVE WESNGQPENN YKTTPPVLDS DGSFFLYSKL TVDKSRWQQG NVFSCSVMHE ALHNHYTQKS LSLSPG (SEQ ID NO:9). Such IgG1 Fc polypeptides may or may not comprise a C-terminal lysine.

In some cases, the Fc polypeptide present in a TMP comprises an amino acid sequence having at least 80%, at least 85%, at least 90%, at least 95%, at least 98%, at least 99%, or 100%, amino acid sequence identity to the following human IgG1 Fc polypeptide amino acid sequence:

(SEQ ID NO: 16)
DKTHTCPPCP APELLGGPSV FLFPPKPKDT LMISRTPEVT CVVVDVSHED PEVKFNWYVD
GVEVHNAKTK PREEQYNSTY RVVSVLTVLH QDWLNGKEYK CKVSNKALPA PIEKTISKAK
GQPREPQVYT LPPSRDELTK NQVSLTCLVK GFYPSDIAVE WESNGQPENN YKTTPPVLDS
DGSFFLYSKL TVDKSRWQQG NVFSCSVMHE ALHNHYTQKS LSLSPGK

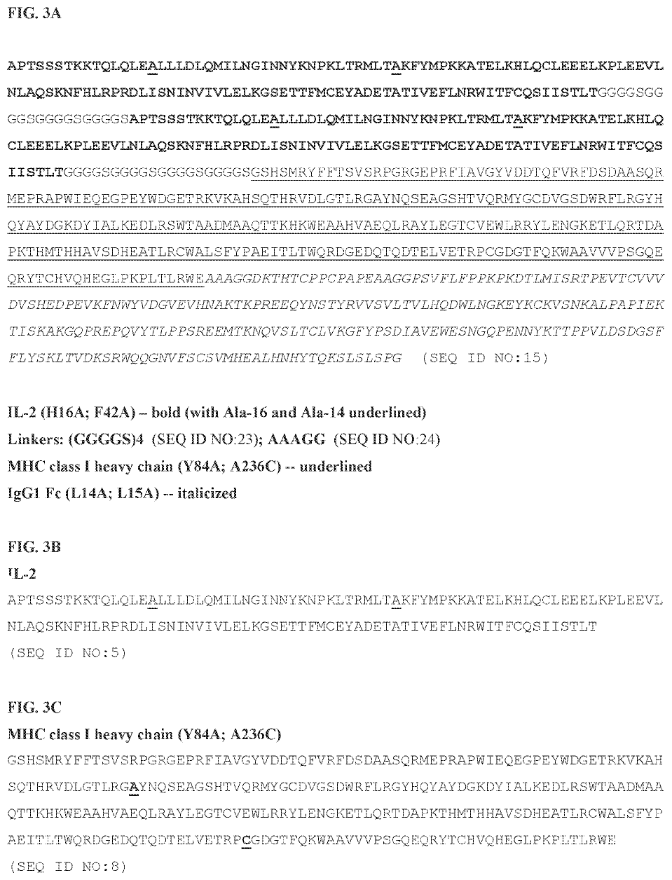

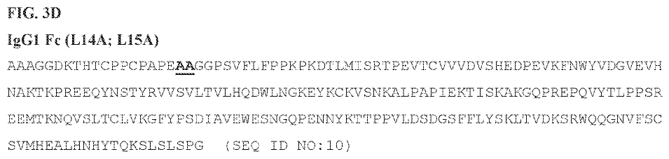

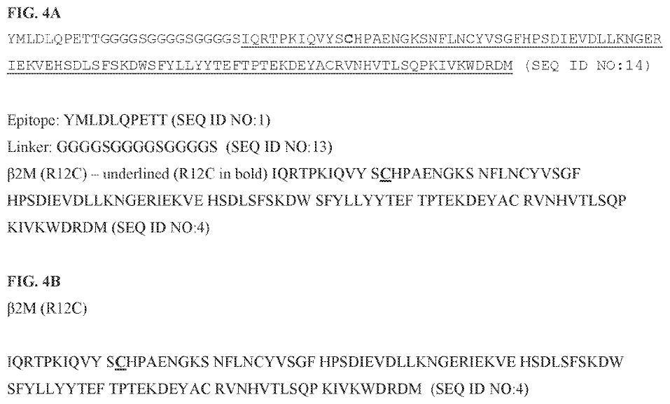

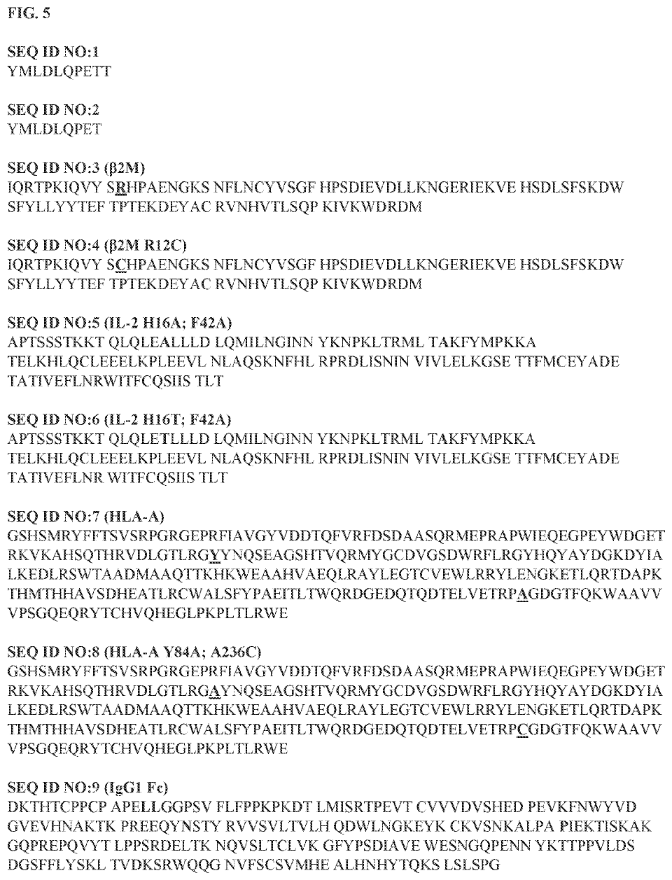

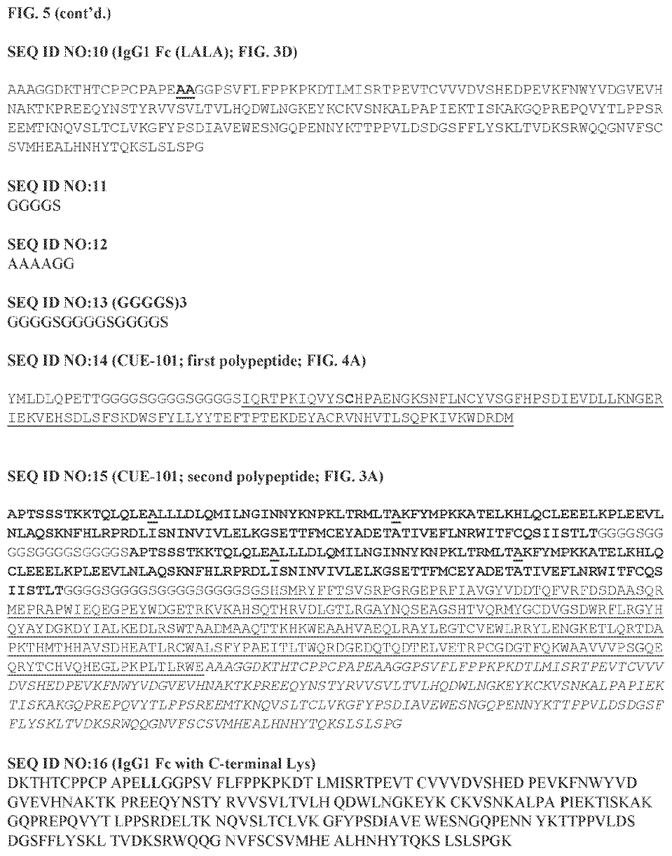

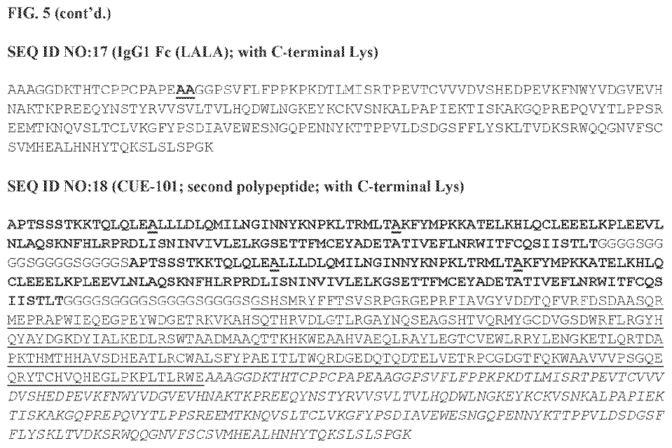

In some cases, the Ig Fc polypeptide present in a TMP comprises an amino acid sequence having at least 80%, at least 85%, at least 90%, at least 95%, at least 98%, at least 99%, or 100%, amino acid sequence identity to the Ig Fc polypeptide amino acid sequence depicted in FIG. 3D, where amino acid 14 is an Ala and amino acid 15 is an Ala. Thus, in some cases, the Ig Fe polypeptide present in a TMP comprises an amino acid sequence having at least 80%, at least 85%, at least 90%, at least 95%, at least 98%, at least 99%, or 100%, amino acid sequence identity to the following Ig Fc amino acid sequence: AAAGGDKTHTCPPCPAPEAAGGPSVFLFPPKPKDTLMI SRTPEVTCVVVDVSHEDPEVKFNWYVDGVEVH NAKTKPREEQYNSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDKSRWQQGNVFSC SVMHEALHNHYTQKSLSLSPG (SEQ ID NO:10), where amino acid 14 is an Ala and amino acid 15 is an Ala.

In some cases, the Ig Fc polypeptide present in a TMP comprises an amino acid sequence having at least 80%, at least 85%, at least 90%, at least 95%, at least 98%, at least 99%, or 100%, amino acid sequence identity to the following Ig Fc amino acid sequence: AAAGGDKTHTCPPCPAPEAAGGPSVFLFPPKPKDTLMI SRTPEVTCVVVDVSHEDPEVKFNWYVDGVEVH NAKTKPREEQYNSTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDKSRWQQGNVFSC SVMHEALHNHYTQKSLSLSPGK (SEQ ID NO:17), where amino acid 14 is an Ala and amino acid 15 is an Ala.

A TMP can include one or more independently selected peptide linkers, i.e., a linker comprising a contiguous stretch of two or more amino acids, where the one or more linkers are between any two components of the TMP, e.g., between one or more of: (i) the peptide epitope and the β2M polypeptide; (ii) a first IL-2 polypeptide and a second IL-2 polypeptide; (iii) an IL-2 polypeptide and an MHC class I heavy chain polypeptide; and (iv) an MHC class I heavy chain polypeptide and an Ig Fc polypeptide. The one or more linkers are independently selected. Suitable linkers include, e.g., (GGGGS)n (SEQ ID NO:29) where n is an integer from 1 to 10 (e.g., n is 1, 2, 3, 4, 5, 6, 7, 8, 9, or 10); AAAGG (SEQ ID NO:24); and the like.

In some cases, a TMP comprises a first polypeptide having at least 90%, at least 95%, at least 98%, at least 99%, or 100%, amino acid sequence identity to the amino acid sequence depicted in FIG. 4A; and a second polypeptide having at least 90%, at least 95%, at least 98%, at least 99%, or 100%, amino acid sequence identity to the amino acid sequence depicted in FIG. 3A.

In some cases, a TMP comprises: a) a first polypeptide having at least 90%, at least 95%, at least 98%, at least 99%, or 100%, amino acid sequence identity to the following amino acid sequence: YMLDLQPETTGGGGSGGGGSGGGGSIQRTPKIQVYSCHPAENGKSNFLNCYVSGFHPSDIEVDLLKNGER IEKVEHSDLSFSKDWSFYLLYYTEFTPTEKDEYACRVNHVTLSQPKIVKWDRDM (SEQ ID NO:14); and
b) a second polypeptide having at least 90%, at least 95%, at least 98%, at least 99%, or 100%, amino acid sequence identity to the following amino acid sequence:

(SEQ ID NO: 15)
APTSSSTKKTQLQLEALLLDLQMILNGINNYKNPKLTRMLTAKFYMPKKATELKHLQCLEEELKPLEEVL
NLAQSKNFHLRPRDLISNINVIVLELKGSETIFMCEYADETATIVEFLNRWITFCQSIISTLTGGGGSGG
GGSGGGGSGGGGSAPTSSSTKKTQLQLEALLLDLQMILNGINNYKNPKLTRMLTAKFYMPKKATELKHLQ
CLEEELKPLEEVLNLAQSKNFHLRPRDLISNINVIVLELKGSETTFMCEYADETATIVEFLNRWITFCQS
IISTLTGGGGSGGGGSGGGGSGGGGSGSHSMRYFFTSVSRPGRGEPRFIAVGYVDDTQFVRFDSDAASQR
MEPRAPWIEQEGPEYWDGETRKVKAHSQTHRVDLGTLRGAYNQSEAGSHTVQRMYGCDVGSDWRFLRGYH
QYAYDGKDYIALKEDLRSWTAADMAAQTTKHKWEAAHVAEQLRAYLEGTCVEWLRRYLENGKETLQRTDA
PKTHMTHHAVSDHEATLRCWALSFYPAEITLTWQRDGEDQTQDTELVETRPCGDGTFQKWAAVVVPSGQE
QRYTCHVQHEGLPKPLTLRWEAAAGGDKTHTCPPCPAPEAAGGPSVFLFPPKPKDILMISRTPEVTCVVV
DVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPG.

In some cases, a TMP comprises: a) a first polypeptide having the following amino acid sequence: YMLDLQPETTGGGGSGGGGSGGGGSIQRTPKIQVYSCHPAENGKSNFLNCYVSGFHPSDIEVDLLKNGER IEKVEHSDLSFSKDWSFYLLYYTEFTPTEKDEYACRVNHVTLSQPKIVKWDRDM (SEQ ID NO:14); and
b) a second polypeptide having the following amino acid sequence:

(SEQ ID NO: 15)
APTSSSTKKTQLQLEALLLDLQMILNGINNYKNPKLTRMLTAKFYMPKKATELKHLQCLEEELKPLEEVL
NLAQSKNFHLRPRDLISNINVIVLELKGSETIFMCEYADETATIVEFLNRWITFCQSIISTLTGGGGSGG
GGSGGGGSGGGGSAPTSSSTKKTQLQLEALLLDLQMILNGINNYKNPKLTRMLTAKFYMPKKATELKHLQ
CLEEELKPLEEVLNLAQSKNFHLRPRDLISNINVIVLELKGSETTFMCEYADETATIVEFLNRWITFCQS
IISTLTGGGGSGGGGSGGGGSGGGGSGSHSMRYFFTSVSRPGRGEPRFIAVGYVDDTQFVRFDSDAASQR
MEPRAPWIEQEGPEYWDGETRKVKAHSQTHRVDLGTLRGAYNQSEAGSHTVQRMYGCDVGSDWRFLRGYH
QYAYDGKDYIALKEDLRSWTAADMAAQTTKHKWEAAHVAEQLRAYLEGTCVEWLRRYLENGKETLQRTDA
PKTHMTHHAVSDHEATLRCWALSFYPAEITLTWQRDGEDQTQDTELVETRPCGDGTFQKWAAVVVPSGQE
QRYTCHVQHEGLPKPLTLRWEAAAGGDKTHTCPPCPAPEAAGGPSVFLFPPKPKDILMISRTPEVTCVVV
DVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPG.

In some cases, a TMP comprises: a) a first polypeptide having the following amino acid sequence:
YMLDLQPETTGGGGSGGGGSGGGGSIQRTPKIQVYSCHPAENGKSNFLNCYVSGFHPSDIEVDLLKNGER IEKVEHSDLSFSKDWSFYLLYYTEFTPTEKDEYACRVNHVTLSQPKIVKWDRDM (SEQ ID NO:14); and
b) a second polypeptide having the following amino acid sequence:

(SEQ ID NO: 18)
APTSSSTKKTQLQLEALLLDLQMILNGINNYKNPKLTRMLTAKFYMPKKATELKHLQCLEEELKPLEEVL
NLAQSKNFHLRPRDLISNINVIVLELKGSETTFMCEYADETATIVEFLNRWITFCQSIISTLIGGGGSGG
GGSGGGGSGGGGSAPTSSSTKKTQLQLEALLLDLQMILNGINNYKNPKLTRMLTAKFYMPKKATELKHLQ
CLEEELKPLEEVLNLAQSKNFHLRPRDLISNINVIVLELKGSETTFMCEYADETATIVEFLNRWITFCQS
IISTLTGGGGSGGGGSGGGGSGGGGSGSHSMRYFFTSVSRPGRGEPRFIAVGYVDDTQFVRFDSDAASQR
MEPRAPWIEQEGPEYWDGETRKVKAHSQTHRVDLGTLRGAYNQSEAGSHTVQRMYGCDVGSDWRFLRGYH
QYAYDGKDYIALKEDLRSWTAADMAAQTTKHKWEAAHVAEQLRAYLEGTCVEWLRRYLENGKETLQRTDA
PKTHMTHHAVSDHEATLRCWALSFYPAEITLTWQRDGEDQTQDTELVETRPCGDGTFQKWAAVVVPSGQE
QRYTCHVQHEGLPKPLTLRWEAAAGGDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALPAPIEK
TISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPCK.

In some cases, the TMP is dimerized. For example, in some cases, one or more interchain disulfide bonds link spontaneous form between two Ig Fe polypeptides (e.g., two IgG1 Fr polypeptides) in two TMPs, thereby forming a homodimer. Thus, in some cases, a TMP is a protein comprising two heterodimers, as described above.

In some cases, a TMP is a homodimer comprising two heterodimers, wherein each heterodimer comprises:
a) a first polypeptide comprising, in order from N-terminus to C-terminus:
   i) an HPV16 E7 epitope comprising the amino acid sequence YMLDLQPETT (SEQ ID NO:1) or YMLDLQPET (SEQ ID NO:2);
   ii) a peptide linker; and
   iii) a β2-microglobulin (β2M) polypeptide comprising the amino acid sequence set forth in SEQ ID NO:4; and
b) a second polypeptide comprising:
   i) two copies of an IL-2 polypeptide, each copy comprising the amino acids sequence set forth in SEQ ID NO: 5;
   ii) a class I MHC heavy chain polypeptide comprising the amino acid sequence set forth in SEQ ID NO: 8; and
   iii) an IgG1 Fc polypeptide comprising the amino acid sequence set forth in SEQ ID NO:10,
wherein the first polypeptide and the second polypeptide of each heterodimer are covalently linked to one another via a disulfide bond between the Cys residue at amino acid 12 of the β2M polypeptide and the Cys residue at amino acid 236 of the class I MHC heavy chain polypeptide, and
wherein the two heterodimers are joined to each other by one or more disulfide bonds that join the IgG1 Fc polypeptides of one heterodimer to the variant immunoglobulin Fc polypeptide of the other heterodimer.

In some cases, a TMP is a homodimer comprising two heterodimers, wherein each heterodimer comprises:
a) a first polypeptide comprising, in order from N-terminus to C-terminus:
   i) an HPV16 E7 epitope comprising the amino acid sequence YMLDLQPETT (SEQ ID NO:1) or YMLDLQPET (SEQ ID NO:2);
   ii) a linker comprising the amino acid sequence GGGGSGGGGSGGGGS (SEQ ID NO:13); and
   iii) a β2-microglobulin (β2M) polypeptide comprising the amino acid sequence set forth in SEQ ID NO:4; and
b) a second polypeptide comprising:
   i) two copies of an IL-2 polypeptide, each copy comprising the amino acids sequence set forth in SEQ ID NO: 5
   ii) an MHC class I heavy chain polypeptide comprising the amino acid sequence set forth in SEQ ID NO: 8; and
   iii) an IgG1 Fc polypeptide comprising an amino acid having a 98% identity to the acid sequence set forth in SEQ ID NO:9 (which does not include a C-terminal Lys) or SEQ ID NO:10 (which does not include a C-terminal Lys) or SEQ ID NO:16 (which does include a C-terminal Lys), or SEQ ID NO:17 (which does include a C-terminal Lys)
wherein the first polypeptide and the second polypeptide of each heterodimer are covalently linked to one another via a disulfide bond between the Cys residue at amino acid 12 of the D2M polypeptide and the Cys residue at amino acid 236 of the class I MHC heavy chain polypeptide, and
wherein the two heterodimers are joined to each other by one or more disulfide bonds that join the gG1 Fc polypeptides of one heterodimer to the variant immunoglobulin Fe polypeptide of the other heterodimer,
wherein the second polypeptide comprises an independently selected peptide linker between one or more of:
   i) the two IL-2 polypeptides;
   ii) one of the two copies of the IL-2 polypeptide and the MHC class I heavy chain polypeptide; and
   iii) the MHC class I heavy chain polypeptide and the IgG1 Fc polypeptide.

In some cases, a TMP is CUE-101, which is a homodimer comprising two heterodimers, wherein each heterodimer comprises:
a) a first polypeptide comprising the amino acid sequence set forth in SEQ ID NO:14;
b) a second polypeptide comprising the amino acid sequence set forth in SEQ ID NO:15 (which includes an Ig Fe polypeptide that does not include a C-terminal Lys) or SEQ ID NO:18 (which includes an Ig Fc polypeptide that does include a C-terminal Lys);
wherein the first polypeptide and the second polypeptide of each heterodimer are covalently linked to one another via a disulfide bond between the Cys residue at amino acid 12 of the β2M polypeptide and the Cys residue at amino acid 236 of the class I MHC heavy chain polypeptide, and
wherein the two heterodimers are joined to each other by one or more disulfide bonds that join the IgG1 Fc polypeptides of one heterodimer to the variant immunoglobulin Fc polypeptide of the other heterodimer.

In some cases, a TMP is a single-chain polypeptide, or a homodimer of two single-chain polypeptides comprising the same amino acid sequence. For example, a TMP may be a single-chain polypeptide comprising (i) an HPV16 E7 epitope comprising the amino acid sequence YMLDLQPETT (SEQ ID NO:1) or YMLDLQPET (SEQ ID NO:2), (ii) a P2-microglobulin (p2M) polypeptide, (iii) one or more copies of an IL-2 polypeptide comprising the amino acids sequence set forth in SEQ ID NO:5, (iv) a class I MHC heavy chain polypeptide, (v) a variant human IgG1 Fe polypeptide, and (vi) one or more independently selected linkers connecting the components of the single-chain polypeptide. The single-chain polypeptide may further comprise one or more disulfide bonds.

For example, a TMP is a homodimer comprising two single-chain polypeptides, wherein each single-chain polypeptide comprises (i) an HPV16 E7 epitope comprising the amino acid sequence YMLDLQPETT (SEQ ID NO:1) or YMLDLQPET (SEQ ID NO:2), (ii) a 32-microglobulin (p2M) polypeptide comprising the amino acid sequence set forth in SEQ ID NO:4, (iii) two copies of an IL-2 polypeptide, each copy comprising the amino acids sequence set forth in SEQ ID NO:5, (iv) an MHC class I heavy chain polypeptide comprising the amino acid sequence set forth in SEQ ID NO: 8; and (v) a variant IgG1 Fc polypeptide comprising an amino acid having a 98% identity to the acid sequence set forth in SEQ ID NO:9 or SEQ ID NO:10,
wherein the two single-chain polypeptides are joined to each other by one or more disulfide bonds that join the variant IgG1 Fc polypeptide of one single-chain polypeptides to the variant IgG1 Fc polypeptide of the other single-chain polypeptide, and
wherein one or more independently selected linkers may connect the components of each single-chain polypeptide.

### Method of Making an MCTL

This disclosure provides a method for making mCTLs. The method comprises introducing one or more nucleic acids into T cells, where the one or more nucleic acids comprise nucleotide sequences encoding (i) a polypeptide (e.g., a CAR) that comprises an antigen-binding domain specific for a cancer-associated antigen, and (ii) an HPV E7_{11-19/11-20} TCR. Methods of introducing such nucleic acids into T cells are known.

This disclosure provides a composition comprising: a) a first nucleic acid (which may be present in a recombinant expression vector) comprising a nucleotide sequence encoding a polypeptide (e.g., a CAR) that comprises an antigen-binding domain specific for a cancer-associated antigen; and b) a second nucleic acid (which may be present in a recombinant expression vector) comprising a nucleotide sequence encoding an HPV E7_{11-19/20} TCR. The present disclosure provides a composition comprising: a) a first recombinant expression vector comprising a nucleotide sequence encoding a polypeptide (e.g., a CAR) that comprises an antigen-binding domain specific for a cancer-associated antigen; and h) a second recombinant expression vector comprising a nucleotide sequence encoding an HPV E7_{11-19/20} TCR. In some cases, the second nucleic acid or the second recombinant expression vector comprises nucleotide sequences encoding the alpha and beta chain amino acid sequences depicted in FIGS. 2A and 2B. In some cases, the second nucleic acid or the second recombinant expression vector comprises nucleotide sequences encoding the alpha and beta chain amino acid sequences depicted in FIGS. 2C and 2D.

A nucleic acid comprising a nucleotide sequence encoding a CAR or a TCR can be present in an expression vector, e.g., recombinant expression vector. Recombinant expression vectors and methods of making recombinant expression vectors are known. In some cases, for example, the recombinant expression vector is a viral construct, e.g., a recombinant adeno-associated virus (AAV) construct, a recombinant adenoviral construct, a recombinant lentiviral construct, a recombinant retroviral construct, etc. In some cases, a nucleic acid comprising a nucleotide sequence encoding a CAR or a TCR is present in a recombinant lentivirus vector. In some cases, a nucleic acid comprising a nucleotide sequence encoding a CAR or a TCR is present in a recombinant AAV vector.

The nucleotide sequence encoding the CAR and/or the nucleotide sequence encoding the TCR can be operably linked to one or more transcriptional control elements, e.g., promoters, such as promoters that are functional in a eukaryotic cell, where the promoter can be a constitutive promoter or an inducible promoter. Suitable promoters are known in the art and any such promoter can be used.

In some cases, the promoter is a CD8 cell-specific promoter, a CD4 cell-specific promoter, a neutrophil-specific promoter, or a natural killer (NK) cell-specific promoter. For example, a CD4 gene promoter can be used; see, e.g., Salmon et al. (1993) Proc. Natl. Acad. Sci. USA 90: 7739; and Marodon et al. (2003) Blood 101:3416. As another example, a CD8 gene promoter can be used.

Any method of introducing nucleic acids into cells can be used to introduce nucleic acid(s) (e.g., recombinant expression vectors) encoding a CAR or encoding a TCR into target T cells. Suitable methods include viral transfection (e.g., where the nucleic acid is a lentiviral vector or other viral vector comprising a nucleotide sequence encoding a CAR or a TCR), electroporation, diethylaminoethyl (DEAE)-dextran-mediated transfection, lipofection, and the like.

In some cases, once the one or more nucleic acids (e.g., recombinant expression vectors) are introduced into T cells in vitro, a mixed population of cells comprising mCTLs and unmodified T cells is generated. In some cases, mCTLs are separated from unmodified T cells. In some cases, the mCTLs comprise at least 80%, at least 85%, at least 90%, at least 95%, at least 98%, or at least 99%, of the total T cell population.

Methods of separating mCTLs from unmodified T cells are known in the art, and any known method can be used. For example, in some cases, a mixed population of cells comprising mCTLs and unmodified T cells is contacted with an immobilized HPV E7 peptide comprising the amino acid sequence YMLDLQPETT (SEQ ID NO:1) or YMLDLQPET (SEQ ID NO:2), allowing the mCTLs to bind to the immobilized HPV E7 peptide, thereby immobilizing the mCTLs, and separating the immobilized mCTLs from unbound cells. In some cases, the immobilized HPV E7 peptide is immobilized on a solid support, such as a bead. In some cases, the immobilized HPV E7 peptide is bound to immobilized MHC class I polypeptides.

In some cases, the method comprises contacting the mCTLs in vitro with a TMP as described herein. Such a contacting step can increase proliferation and/or activation of the mCTLs. In some cases, contacting the mCTLs with a TMP increases proliferation of the mCTLs by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the level of proliferation in the absence of the TMP (i.e., the level of proliferation of mCTLs not contacted with the TMP). In some cases, following contact with a TMP, the number of mCTLs increases by at least 10%, at least 15%, at least 20%, at least 25%, at least 30%, at least 35%, at least 40%, at least 45%, at least 50%, at least 75%, at least 100% (or 2-fold), at least 2.5-fold, at least 5-fold, at least 10-fold, at least 50-fold, at least 100-fold, or more than 100-fold, compared to the number of mCTLs not contacted with the TMP.

### Treatment Methods

The present disclosure provides methods of treating cancer in an individual. The methods generally involve administering to an individual having a cancer a composition comprising mCTLs of the present disclosure. In some cases, the method comprises: a) introducing into an individual having a cancer a composition comprising mCTLs and a composition comprising a TMP as described herein. In some cases, the method comprises: a) introducing into an individual having a cancer a composition comprising mCTLs; and b) administering to the individual a TMP as described herein.

A method of the present disclosure comprises administering an effective amount of mCTLs. An effective amount of mCTLs is an amount that, when administered in one or more doses to an individual in need thereof, either as a monotherapy or as part of a combination therapy (e.g., as part of a combination therapy with a TMP and/or an immune checkpoint inhibitor, as discussed below), reduces the overall tumor burden in the individual, i.e., the amount of cancer in the body, or alternatively, causes the total tumor burden in the patient to remain relatively stable for a sufficient period of time for the patient to have a confirmed “stable disease” as determined by standard RECIST criteria.

In some cases, an effective amount of mCTLs is an amount that, when administered in one or more doses to an individual in need thereof, either as a monotherapy or as part of a combination therapy (e.g., as part of a combination therapy with a TMP and/or an immune checkpoint inhibitor), causes the tumor size to be reduced by a sufficient amount, and for a sufficient period of time, for the patient to have a confirmed “partial response” as determined by standard RECIST criteria.

In some cases, an effective amount of mCTLs is an amount that, when administered in one or more doses to an individual in need thereof, either as a monotherapy or as part of a combination therapy (e.g., as part of a combination therapy with a TMP and/or an immune checkpoint inhibitor), causes the tumor size to be reduced by a sufficient amount, and for a sufficient period of time, for the patient to have a confirmed “complete response” as determined by standard RECIST criteria.

In some cases, an effective amount of mCTLs is an amount that, when administered in one or more doses to an individual in need thereof (an individual having a tumor), either as a monotherapy or as part of a combination therapy (e.g., as part of a combination therapy with a TMP and/or an immune checkpoint inhibitor), reduces the tumor volume in the individual. For example, in some cases, an effective amount of mCTLs is an amount that, when administered in one or more doses to an individual in need thereof (an individual having a tumor), either as a monotherapy or as part of a combination therapy (e.g., as part of a combination therapy with a TMP and/or an immune checkpoint inhibitor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the mCTLs, or in the absence of administration with the mCTLs. Tumor volume is determined using the formula (length×width×width)/2, where length represents the largest tumor diameter and width represents the perpendicular tumor diameter.

In some cases, an effective amount of mCTLs is an amount that, when administered in one or more doses to an individual in need thereof, increases survival time of the individual. For example, in some cases, an effective amount of mCTLs is an amount that, when administered in one or more doses to an individual in need thereof, either as a monotherapy or as part of a combination therapy (e.g., as part of a combination therapy with a TMP and/or an immune checkpoint inhibitor), increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the mCTLs.

In some cases, an effective amount of mCTLs is an amount that, when administered in one or more doses to an individual in need thereof, either as a monotherapy or as part of a combination therapy (e.g., as part of a combination therapy with a TMP and/or an immune checkpoint inhibitor), reduces the level of circulating tumor DNA (“ctDNA”) in the patient by at least 10%, at least 15%, at least 20%, at least 25%, at least 30%, at least 40%, at least 50%, at least 60%, at least 70%, at least 80%, at least 90%, or at least 95%, compared to the ctDNA levels in the individual before administration of the mCTLs, or in the absence of administration with the mCTLs. The level of ctDNA can be determined using any known method; see, e.g., Cescon et al. (2020) Nature Cancer 1:276.

In some cases, a method of the present disclosure comprises: a) contacting mCTLs in vitro with a TMP; and b) administering to an individual in need thereof an effective amount of a composition comprising the mCTLs in admixture with the TMP. In some cases, the contacting step is carried out for a period of time to allow for proliferation of the mCTLs in vitro. For example, in some cases, the contacting step is carried out for a period of time of from 1 hour to 36 hours (e.g., from 1 hour to 2 hours, from 2 hours to 4 hours, from 4 hours to 8 hours, from 8 hours to 12 hours, from 12 hours to 18 hours, from 18 hours to 24 hours, or from 24 hours to 36 hours, or longer than 36 hours before the administering step.

In some cases, a method of the present disclosure comprises: a) administering an effective amount of mCTLs to an individual in need thereof; and b) administering to the individual an effective amount of a TMP. In some cases, the TMP is administered prior to administration of the mCTLs. In some cases, the TMP is administered prior to and after administration of the mCTLs. In some cases, the TMP is administered prior to and following administration of the mCTLs.

In some cases, the TMP is administered after administration of the mCTLs. e.g., from about 1 hour to about 1 week after step administration of the mCTLs. For example, in some cases, step (b) is carried out at a period of time of from 1 hour to about 2 hours, from 2 hours to 4 hours, from 4 hours to 8 hours, from 8 hours to 12 hours, from 12 hours to 18 hours, from 18 hours to 24 hours, from 24 hours to 2 days, from 2 days to 4 days, or from 4 days to 1 week, after step (a). Thereafter, the TMP may be re-administered periodically, as determined by the patient's physician, to maintain the desired level of mCTLs in the patient. For example, the TMP may be administered every week, every two weeks, every three weeks, monthly, or less frequently than monthly. The TMP also may be administered more frequently following the initial administration of mCTLs, and then less frequently once the desired level of mCTLs cells has been reached.

In some cases, a method of the present disclosure comprises administering mCTLs in an amount of from 10 cells/kg body weight to 10⁹ cells/kg body weight. For example, in some cases, a method of the present disclosure comprises administering mCTLs in an amount of from 10 cells/kg body weight to 10² cells/kg body weight, from 10² cells/kg body weight to 10³ cells/kg body weight, from 10³ cells/kg body weight to 10⁴ cells/kg body weight, from 10⁴ cells/kg body weight to 10⁵ cells/kg body weight, from 10⁵ cells/kg body weight to 10⁶ cells/kg body weight, from 10⁶ cells/kg body weight to 10⁷ cells/kg body weight, from 10⁷ cells/kg body weight to 10⁸ cells/kg body weight, or from 10⁸ cells/kg body weight to 10⁹ cells/kg body weight. In some cases, a suitable number of mCTLs is equal to or less than a number selected from the group consisting of 10 cells/kg body weight, 10² cells/kg body weight, 10³ cells/kg body weight, 10⁴ cells/kg body weight, 10⁵ cells/kg body weight, 10⁶ cells/kg body weight, 10⁷ cells/kg body weight, 10⁸ cells/kg body weight and 10⁹ cells/kg body weight. In some cases, the method comprises administering a composition comprising a quantity of mCTLs that is equal to or less than 10⁷ cells/kg body weight. In some cases, the method comprises administering a composition comprising a quantity of mCTLs that is equal to or less than 10⁶ cells/kg body weight. In some cases, the method comprises administering a composition comprising a quantity of mCTLs that is equal to or less than 10⁵ cells/kg body weight.

Where a method of the present disclosure comprises administering a TMP, the TMP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. For example, a TMP can be administered in an amount of from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight. If the regimen is a continuous infusion, it can also be in the range of 1 μg to 10 mg per kilogram of body weight per minute.

Where TMPs as described above are employed, e.g., a homodimer comprising two heterodimers, wherein each heterodimer comprises two copies of an IL-2 polypeptide, each copy comprising the amino acids sequence set forth in SEQ ID NO:5, such homodimers may be administered in an amount of 4 mg/kg body weight or less, e.g., 3 mg/kg body weight, 2 mg/kg body weight, 1 mg/kg body weight, or less than 1 mg/kg body weight, with the amount being determined by the patient's physician to maintain the desired level of mCTLs in the patient.

Generally speaking, when a combination therapy of mCTLs and TMPs is employed, the mCTLs may be administered in an amount that is at least one order of magnitude lower than the dose that would be in the absence of administration of the TMP, i.e., because the TMP is then able to increase the number of mCTLs in the patient. In some cases, the mCTLs may be administer in an amount that is at least two orders of magnitude lower than the dose that would be in the absence of administration of the TMP. In some cases, the mCTLs may be administer in an amount that is at least three orders of magnitude lower than the dose that would be in the absence of administration of the TMP.

For example, according to the FDA approved package insert, where CAR-T cells are genetically modified to produce Yescarta®, the number of such CAR-T cells that are administered in a monotherapy regimen to an individual having a CD19⁺ cancer (e.g., a B cell lymphoma such as large B-cell lymphoma of follicular lymphoma) is about 2×10⁶ CAR-positive viable T cells per kg body weight up to a maximum of 2×10⁸ CAR-positive viable T cells. In a combination therapy with a TMP, however, a lesser number of Yescarta® CAR-T cells may be administered to the patient, e.g. one order of magnitude less, e.g., about 2×10⁵ CAR-positive viable T cells per kg body weight up to a maximum of 2×10⁷ CAR-positive viable T cells, about 2×10⁴ CAR-positive viable T cells per kg body weight up to a maximum of 2×10⁶ CAR-positive viable T cells, or about 2×10³ CAR-positive viable T cells per kg body weight up to a maximum of 2×10⁵ CAR-positive viable T cells. In this way, the necessity for lymphodepletion prior to administration of the Yescarta® CAR-T cells can be reduced or substantially eliminated. Likewise, the need for treatment with recombinantly produced IL-2, e.g., aldesleukin (Proleukin®), following administration of the Yescarta® CAR-T cells can also be reduced or substantially eliminated. The initial administration of fewer Yescarta® CAR-T cells also can reduce one or more of the adverse side effects (e.g., cytokine release syndrome and/or neurological toxicities) associated with the administration of such high numbers of Yescarta® CAR-T cells. Similar results and benefits may be achieved by administering a TMP with lower amounts, e.g., at least one order of magnitude, at least two orders of magnitude, or at least three orders of magnitude fewer numbers of CAR-T cells than the number indicated in the FDA approved package insert, for other CAR-T therapies such as Tecartus®, Kymriah®, Abecma® or Breyanzi®, as well as future approved CAR-T therapies.

In some cases, a method of the present disclosure for treating a cancer does not comprise a lymphodepleting regimen. In other words, in some cases, an individual being treated with mCTLs has not undergone lymphodepleting chemotherapy. Lymphodepleting chemotherapy can comprise administration of, e.g., cyclophosphamide and fludarabine before infusion of T cells (e.g., from 2 days to 7 days before infusion of T cells).

In some cases, a method of the present disclosure for treating a cancer does not comprise administering recombinantly produced IL-2, e.g., aldesleukin (Proleukin®), following administration of the mCTLs.

In some cases, a method of the present disclosure for treating a cancer does not comprise (i) a lymphodepleting regimen, or (ii) administering recombinantly produced IL-2, e.g., aldesleukin (Proleukin®), following administration of the mCTLs.

A quantity of mCTLs can be administered to an individual in need thereof via any of a variety of routes of administration. For example, a composition comprising mCTLs can be administered via an intramuscular, an intravenous, a peritumoral, or an intratumoral route of administration. Similarly, where a TMP is administered to the individual, a TMP can be administered to the individual via any of a variety of routes of administration. For example, a composition comprising a TMP (or a dimerized TMP) can be administered via an intramuscular, an intravenous, a peritumoral, or an intratumoral route of administration.

### Combination Therapy

In some cases, a method of the present disclosure for treating cancer in an individual comprises: a) administering mCTLs to an individual; and b) administering at least one additional therapeutic agent or therapeutic treatment to the individual. In some cases, a method of the present disclosure for treating cancer in an individual comprises: a) administering mCTLs and a TMP to an individual; and b) administering at least one additional therapeutic agent or therapeutic treatment to the individual. In some cases, a method of the present disclosure for treating cancer in an individual comprises: a) administering mCTLs and a TMP to an individual; b) administering a TMP to the individual; and c) administering at least one additional therapeutic agent or therapeutic treatment to the individual. Suitable additional therapeutic agents include, but are not limited to, a small molecule cancer chemotherapeutic agent, and an immune checkpoint inhibitor. Suitable additional therapeutic treatments include, e.g., radiation, surgery (e.g., surgical resection of a tumor), and the like.

A treatment method of the present disclosure can comprise co-administration of a CART-MP and at least one additional therapeutic agent. By “co-administration” is meant that both a CART-MP and at least one additional therapeutic agent are administered to an individual, although not necessarily at the same time, in order to achieve a therapeutic effect that is the result of having administered both the CART-MP and the at least one additional therapeutic agent. The administration of the CART-MP and the at least one additional therapeutic agent can be substantially simultaneous, e.g., the CART-MP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a CART-MP is administered to an individual who is undergoing treatment with, or who has undergone treatment with, the at least one additional therapeutic agent. The administration of the CART-MP can occur at different times and/or at different frequencies.

As an example, a treatment method of the present disclosure can comprise co-administration of a CART-MP and an immune checkpoint inhibitor such as an antibody specific for an immune checkpoint. By “co-administration” is meant that both a CART-MP and an immune checkpoint inhibitor (e.g., an antibody specific for an immune checkpoint polypeptide) are administered to an individual, although not necessarily at the same time, in order to achieve a therapeutic effect that is the result of having administered both the CART-MP and the immune checkpoint inhibitor (e.g., an antibody specific for an immune checkpoint polypeptide). The administration of the CART-MP and the immune checkpoint inhibitor (e.g., an antibody specific for an immune checkpoint polypeptide) can be substantially simultaneous, e.g., the CART-MP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, within about 24 hours, within 1 week, 3 weeks 3 weeks, four weeks or a month following administration of the immune checkpoint inhibitor (e.g., an antibody specific for an immune checkpoint polypeptide). In some cases, a CART-MP of the present disclosure is administered to an individual who is undergoing treatment with, or who has undergone treatment with, an immune checkpoint inhibitor (e.g., an antibody specific for an immune checkpoint polypeptide). The administration of the CART-MP and the immune checkpoint inhibitor (e.g., an antibody specific for an immune checkpoint polypeptide) can occur at different times and/or at different frequencies. Where there is an established dosing interval for the checkpoint inhibitor, depending on the interval, it may be possible to administer the CART-MP on the same day as the checkpoint inhibitor. For example, in some cases, where the dosing schedule for pembrolizumab is once every three weeks, the pharmaceutical composition comprising the CART-MP may be administered on the same day.

Exemplary immune checkpoint inhibitors include inhibitors that target an immune checkpoint polypeptide such as CD27, CD28, CD40, CD122, CD96, CD73, CD47, OX40, GITR, CSFIR, JAK, PI3K delta, PI3K gamma, TAM, arginase, CD137 (also known as 4-1BB), ICOS, A2AR. B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122 and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint polypeptide. In some cases, the anti-immune checkpoint antibody is a monoclonal antibody. In some cases, the anti-immune checkpoint antibody is humanized, or de-immunized such that the antibody does not substantially elicit an immune response in a human. In some cases, the anti-immune checkpoint antibody is a humanized monoclonal antibody. In some cases, the anti-immune checkpoint antibody is a de-immunized monoclonal antibody. In some cases, the anti-immune checkpoint antibody is a fully human monoclonal antibody. In some cases, the anti-immune checkpoint antibody inhibits binding of the immune checkpoint polypeptide to a ligand for the immune checkpoint polypeptide. In some cases, the anti-immune checkpoint antibody inhibits binding of the immune checkpoint polypeptide to a receptor for the immune checkpoint polypeptide.

Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urclumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, and AMP-224. In some cases, the anti-PD-1 monoclonal antibody is nivolumab, pembrolizumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some cases, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149. In some cases, the immune checkpoint inhibitor is an anti-TIGIT antibody that binds to T-cell immunoreceptor with immunoglobulin and ITIM domains (TIGIT). In some cases, the anti-TIGIT antibody is BMS-986207 (Bristol-Myers Squibb). In some cases, the anti-TIGIT antibody is tiragolumab. In some cases, the anti-TIGIT antibody is EOS88448 (EOS-448). See, e.g., U.S. Pat. Nos. 11,008,390 and 10,189,902; U.S. Patent Publication No. 2017/0088613; and WO 2019/137541.

Among such checkpoint inhibitors, antibodies to PD-1, PD-L1, TIGIT, LAG3 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. A quantity of mCTLs of this disclosure also may be co-administered with combinations of checkpoint inhibitors, e.g., a combination of (i) an antibody to PD-1 or PD-L1, and (ii) an antibody to CTLA-4.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment include individuals having a cancer in which the cancer cells express, or overexpress, a cancer-associated antigen.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

### Aspects Set 1

Aspect 1. A quantity of modified cytotoxic T cells (“mCTLs”), wherein the mCTLs comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2) (an “HPV E7_{11-19/20} TCR”; and b) one or more nucleic acids comprising nucleotide sequences encoding a chimeric antigen receptor (CAR), wherein the CAR comprises an antigen-binding domain specific for a cancer-associated antigen, and wherein the percentage of target mCTLs cells in the composition exceeds at least 1% of the total number of T cells in the composition.

Aspect 2. A quantity of modified cytotoxic T cells (“mCTLs”), wherein the mCTLs comprise: a) an HPV E7_{11-19/20} TCR; and b) a chimeric antigen receptor (CAR), wherein the CAR comprises an antigen-binding domain specific for a cancer-associated antigen, and wherein the percentage of target mCTLs cells in the composition exceeds at least 1% of the total number of T cells in the composition.

Aspect 3. A quantity of modified cytotoxic T cells (“mCTLs”), wherein the mCTLs comprise: a) a heterologous T-cell receptor that is a an HPV E7_{11-19/20} TCR; and b) a chimeric antigen receptor (CAR), wherein the CAR comprises an antigen-binding domain specific for a cancer-associated antigen.

Aspect 4. A quantity of mCTLs according to any one of aspects 1-3, wherein the CAR comprises: a) an extracellular domain comprising the antigen-binding domain; b) a transmembrane region; and c) a cytoplasmic domain comprising an intracellular signaling domain.

Aspect 5. A quantity of mCTLs according to aspect 4, wherein the cytoplasmic domain comprises one or more costimulatory polypeptides.

Aspect 6. A quantity of mCTLs according to aspect 5, wherein the costimulatory polypeptide is selected from CD28, 4-1BB, and OX-40.

Aspect 7. A composition according to any one of aspects 4-6, wherein the intracellular signaling domain comprises a signaling domain from the zeta chain of human CD3.

Aspect 8. A quantity of mCTLs according to any one of aspects 1-7, wherein the antigen-binding domain is a single-chain Fv polypeptide or a nanobody.

Aspect 9. A quantity of mCTLs according to any one of aspects 1-8, wherein the CAR is a single polypeptide chain CAR.

Aspect 10. A quantity of mCTLs according to any one of aspects 1-8, wherein the CAR comprises at least two polypeptide chains.

Aspect 11. A quantity of mCTLs according to any one of aspects 1-10, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR. EGFRVIII, EpCAM, EphA2, GD-2, glypican-3, GPC3. HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 12. A quantity of mCTLs according to any one of aspects 1-11, wherein the TCR comprises: (i) an α chain having at least 90% amino acid sequence identity to the α-chain amino acid sequence depicted in FIG. 2A; and (ii) a having at least 90% amino acid sequence identity to the β chain amino acid sequence depicted in FIG. 2B.

Aspect 13. A quantity of mCTLs according to any one of aspects 1-11, wherein the TCR comprises: (i) an α chain having at least 90% amino acid sequence identity to the α-chain amino acid sequence depicted in FIG. 2C; and (ii) a having at least 90% amino acid sequence identity to the β chain amino acid sequence depicted in FIG. 2D.

Aspect 14. A quantity of mCTLs according to any one of aspects 1-13, wherein the mCTLs are CD8+ T cells.

Aspect 15. A quantity of mCTLs according to any one of aspects 1-13, wherein the mCTLs arc killer T cells or killer innate-like T cells.

Aspect 16. A composition comprising a quantity of T cells, wherein the quantity of T cells comprises mCTLs according to any one of aspects 1-15, and wherein the percentage of T cells that are mCTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%,
optionally wherein the composition is a pharmaceutical composition suitable for administration into a human subject.

Aspect 17. An admixture comprising
(i) a quantity of mCTLs according to any one of aspects 1-15 or a composition according to aspect 16; and
(ii) a quantity of a homodimer comprising two heterodimers, wherein each heterodimer comprises:
a) a first polypeptide comprising:
   i) a human papillomavirus epitope, wherein the epitope comprises the amino acid sequence YMLDLQPETT (SEQ ID NO:1) or YMLDLQPET (SEQ ID NO:2); and
   ii) a first major histocompatibility complex (MHC) polypeptide, wherein the first MHC polypeptide is a B2-microglobulin (B2M) polypeptide comprising an amino acid sequence as depicted in FIG. 4B, and
b) a second polypeptide comprising:
   i) two copies of an IL-2 polypeptide, each copy comprising an amino acid sequence as depicted in FIG. 3B;
   iii) a second MHC polypeptide, wherein the second MHC polypeptide is an MHC class I heavy chain polypeptide comprising an amino acid sequence as depicted in FIG. 3C; and
   iv) an immunoglobulin (Ig) Fc polypeptide,
wherein the first polypeptide and the second polypeptide are covalently linked to one another via a disulfide bond.

Aspect 18. An admixture according to aspect 17,
wherein the Ig Fc polypeptide comprises an amino acid sequence having at least about 95% percent amino acid sequence identity to the amino acid sequence depicted in FIG. 3D, wherein the percent sequence identity is determinable by a sequence alignment performed using BLAST, and wherein the Ig Fc polypeptide comprises a Leu at position 14 and a Leu at position 15 based on the amino acid numbering depicted FIG. 3D,
wherein the first and second polypeptides are covalently linked to one another via a disulfide bond between the Cys residue at amino acid 12 of the β2M polypeptide and the Cys residue at amino acid 236 of the class I MHC heavy chain polypeptide,
wherein the first polypeptide comprises a peptide linker between the epitope and the β2M polypeptide, and wherein the second polypeptide comprises a peptide linker between one or more of:
a) a first copy of the IL-2 polypeptide and a second copy of the IL-2 polypeptide;
b) one of the two copies of the IL-2 polypeptide and the MHC class I heavy chain polypeptide; and
c) the MHC class I heavy chain polypeptide and the Ig Fc polypeptide,
optionally wherein the homodimer comprises two heterodimers, wherein each heterodimer comprises a first polypeptide comprising the amino acid sequence set forth in SEQ ID NO:14, and a second polypeptide comprising the amino acid sequence set forth in SEQ ID NO:15 or SEQ ID NO:18.

Aspect 19. A method of making a quantity of mCTLs according to any one of aspects 1-15, wherein the method comprises modifying a quantity of T cells by introducing one or more nucleic acids encoding
(i) a chimeric antigen receptor (CAR) that comprises an antigen-binding domain specific for a cancer-associated antigen, and
(ii) an HPV E7_{11-19/20} TCR.

Aspect 20. A method according to aspect 19, wherein the method comprises the step of at least partially separating target T cells comprising an HPV E7_{11-19/20} TCR, optionally wherein the step of at least partially separating target T cells comprising an HPV E7_{11-19/20} TCR comprises binding the target T cells to an HPV16 E7 peptide comprising the amino acid sequence YMLDLQPETT (SEQ ID NO:1) or YMLDLQPET (SEQ ID NO:2).

Aspect 21. A method according to aspect 20, wherein the HPV16 E7 peptide is immobilized on an insoluble support.

Aspect 22. A method according to aspect 21, wherein insoluble support is a bead.

Aspect 23. A method according to aspect 19, wherein the HPV16 peptide is a peptide-loaded MHC class I multimer.

Aspect 24. A method according to any one of aspects 19-23, wherein the CAR comprises: a) an extracellular domain comprising the antigen-binding domain; b) a transmembrane region; and c) a cytoplasmic domain comprising an intracellular signaling domain.

Aspect 25. A method according to aspect 24, wherein the intracellular signaling domain comprises a signaling domain from the zeta chain of human CD3.

Aspect 26. A method according to aspect 24 or 25, wherein the cytoplasmic domain comprises one or more costimulatory polypeptides.

Aspect 27. A method according to aspect 26, wherein the costimulatory polypeptide is selected from CD28, 4-1BB, and OX-40.

Aspect 28. A method according to any one of aspects 19-27, wherein the antigen-binding domain is a single-chain Fv polypeptide or a nanobody.

Aspect 29. A method according to any one of aspects 19-28, wherein the CAR is a single polypeptide chain CAR.

Aspect 30. A method according to any one of aspects 19-28, wherein the CAR comprises two polypeptide chains.

Aspect 31. A method according to any one of aspects 19-30, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRVIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 32. A method according to any one of aspects 19-31, wherein the percentage of total number of T cells in the composition that are target mCTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

Aspect 33. A method according to any one of aspects 19-32, wherein prior to step (ii), the composition comprising a quantity of T cells is contacted, in vitro or in vivo, with a TMP, wherein the TMP comprises a heterodimeric polypeptide comprising:
a) a first polypeptide comprising: (i) an HPV16 peptide comprising the amino acid sequence YMLDLQPETT (SEQ ID NO:1) or YMLDLQPET (SEQ ID NO:2); (ii) a linker; and (iii) a β2 microglobulin polypeptide; and
b) a second polypeptide comprising: (i) two copies of an IL-2 polypeptide; (ii) an MHC class I heavy chain polypeptide; and (iii) an immunoglobulin (Ig) Fc polypeptide,
optionally wherein the TMP comprises a homodimer comprising two heterodimeric polypeptides, wherein each heterodimeric polypeptide comprises a first polypeptide comprising the amino acid sequence set forth in SEQ ID NO:14, and a second polypeptide comprising the amino acid sequence set forth in SEQ ID NO:15 or SEQ ID NO:18.

Aspect 34. A method according to any one of aspects 19-33, wherein at least 50% of the target T cells are CD8+ T cells.

Aspect 35. A method according to any one of aspects 19-34, comprising, between steps (i) and (ii), enriching the T cells for CD8+ T cells.

Aspect 36. A method according to any one of aspects 19-35, comprising, between steps (ii) and (iii), enriching the T cells for CD8+ T cells.

Aspect 37. A method of treating a cancer in an individual, the method comprising introducing into the individual a composition comprising a quantity of modified cytotoxic T cells according to any one of aspects 1-15, or a pharmaceutical composition prepared according to the method of any one of aspects 16-33.

Aspect 38. A method of according to aspect 37, further comprising administering to the individual a composition comprising a TMP comprising either:
a) heterodimeric polypeptide comprising
a first polypeptide comprising: (i) an HPV16 peptide comprising the amino acid sequence YMLDLQPETT (SEQ ID NO:1) or YMLDLQPET (SEQ ID NO:2); (ii) a linker; and (iii) a β2 microglobulin polypeptide; and
a second polypeptide comprising: (i) two copies of an IL-2 polypeptide; (ii) an MHC class I heavy chain polypeptide; and (iii) a variant immunoglobulin (Ig) Fc polypeptide,
optionally wherein the TMP comprises a homodimer comprising two heterodimeric polypeptides, wherein each heterodimeric polypeptide comprises a first polypeptide comprising the amino acid sequence set forth in SEQ ID NO:14, and a second polypeptide comprising the amino acid sequence set forth in SEQ ID NO:15 or SEQ ID NO:18;
or
b) a single-chain polypeptide comprising: (i) an HPV 16 peptide comprising the amino acid sequence YMLDLQPETT (SEQ ID NO:1) or YMLDLQPET (SEQ ID NO:2), (ii) a β2 microglobulin polypeptide, (iii) one or more copies of a variant IL-2 polypeptide; (iv) an MHC class I heavy chain polypeptide, and (v) a variant immunoglobulin (Ig) Fc polypeptide.

Aspect 39. A method according to aspect 37 or aspect 38, wherein said administering a composition comprising a quantity of genetically modified cytotoxic T cells comprises administering a quantity of genetically modified cytotoxic T cells that is equal to or less than a number selected from the group consisting of 10 cells/kg body weight, 10² cells/kg body weight, 10³ cells/kg body weight, 10⁴ cells/kg body weight, 10⁵ cells/kg body weight, 10⁶ cells/kg body weight, 10⁷ cells/kg body weight, 10⁸ cells/kg body weight and 10⁹ cells/kg body weight.

Aspect 40. A method according to any of aspects 37-39, wherein said administering a composition comprising a quantity of genetically modified cytotoxic T cells comprises administering a quantity of genetically modified cytotoxic T cells that is equal to or less than 10⁷ cells/kg body weight.

Aspect 41. A method according to any of aspects 37-40, wherein the individual does not undergo a lymphodepleting regimen prior to the introducing step.

Aspect 42. A method according to any one of aspects 37-41, wherein said administering is intramuscular, intravenous, peritumoral, or intratumoral.

Aspect 43. A method according to any one of aspects 37-42, further comprising administering one or more checkpoint inhibitors to the individual.

Aspect 44. A method according to aspect 43, wherein the checkpoint inhibitor is an antibody that binds to a polypeptide selected from the group consisting of CD27, CD28, CD40, CD122, CD96, CD73, CD47, OX40, GITR, CSFIR, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1, and PD-L2.

Aspect 45. A method according to aspect 43, wherein the checkpoint inhibitor is an antibody specific for PD-1, PD-L1, or CTLA4.

Aspect 46. A method according to aspect 45, wherein the one or more checkpoint inhibitors is selected from the group consisting of nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, Mogamulizumab, Varlilumab, Avelumab, Galiximab, AMP-514, AUNP 12, Indoximod, NLG-919, INCB024360, KN035, and combinations thereof.

Aspect 47. A quantity of modified cells according to any one of aspects 1-15, wherein instead of the HPV E7_{11-19/20} TCR, the modified cells comprise a T-cell receptor (TCR) specific for MHC class I polypeptides that present peptide other than a human papilloma virus (HPV) E7 peptide comprising the amino acid sequence YMLDLQPETT or YMLDLQPET.

Aspect 48. A quantity of modified cells according to any one of aspects 1-15 or 47, wherein instead of a CAR, the modified cells comprise an antigen-binding domain other than a CAR.

Aspect 48. A quantity of modified cells according to any one of aspects 1-15, 47 or 48, wherein instead of T cells, the modified cells are cells other than T cells, e.g., NK cells.

### Aspects Set 2

Aspect 1. A quantity of modified cytotoxic cells (“mCLs”), wherein the mCLs comprise: a) one or more nucleic acids comprising nucleotide sequences encoding a predetermined T-cell receptor (TCR); and b) an antigen-binding domain specific for a cancer-associated antigen, and wherein the percentage of target mCLs cells in the composition exceeds at least 1% of the total number of T cells in the composition.

Aspect 2. A quantity of modified cytotoxic cells (“mCLs”), wherein the mCLs comprise: a) an HPV E7_{11-19/20} TCR; and h) an antigen-binding domain specific for a cancer-associated antigen, and wherein the percentage of target mCLs cells in the composition exceeds at least 1% of the total number of T cells in the composition, optionally wherein the antigen binding domain in a heterologous antigen-binding domain.

Aspect 3. A quantity of modified cytotoxic cells (“mCLs”), wherein the mCLs comprise: a) a heterologous T-cell receptor that is a an HPV E7_{11-19/20} TCR; and b) a heterologous antigen-binding domain specific for a cancer-associated antigen.

Aspect 4. A quantity of mCLs according to any one of aspects 1-3, wherein the mCLs comprise a CAR, and wherein the comprises: a) an extracellular domain comprising the antigen-binding domain; b) a transmembrane region; and c) a cytoplasmic domain comprising an intracellular signaling domain.

Aspect 5. A quantity of mCLs according to aspect 4, wherein the cytoplasmic domain comprises one or more costimulatory polypeptides.

Aspect 6. A quantity of mCLs according to aspect 5, wherein the costimulatory polypeptide is selected from CD28, 4-1BB, and OX-40.

Aspect 7. A composition according to any one of aspects 4-6, wherein the intracellular signaling domain comprises a signaling domain from the zeta chain of human CD3.

Aspect 8. A quantity of mCLs according to any one of aspects 1-7, wherein the antigen-binding domain is a single-chain Fv polypeptide or a nanobody.

Aspect 9. A quantity of mCLs according to any one of aspects 1-8, wherein the mCLs comprise a CAR, and wherein the CAR is a single polypeptide chain CAR.

Aspect 10. A quantity of mCLs according to any one of aspects 1-8, wherein the mCLs comprise a CAR, and wherein the CAR comprises at least two polypeptide chains.

Aspect 11. A quantity of mCLs according to any one of aspects 1-10, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRVIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 12. A quantity of mCLs according to any one of aspects 1-11, wherein the TCR comprises: (i) an α chain having at least 90% amino acid sequence identity to the α-chain amino acid sequence depicted in FIG. 2A; and (ii) a having at least 90% amino acid sequence identity to the β chain amino acid sequence depicted in FIG. 2B.

Aspect 13. A quantity of mCLs according to any one of aspects 1-11, wherein the TCR comprises: (i) an α chain having at least 90% amino acid sequence identity to the α-chain amino acid sequence depicted in FIG. 2C; and (ii) a having at least 90% amino acid sequence identity to the β chain amino acid sequence depicted in FIG. 2D.

Aspect 14. A quantity of mCLs according to any one of aspects 1-13, wherein the mCLs NK cells, macrophages or ILC (innate lymphoid cells), and wherein the cells a natural, modified or heterologous antigen-binding domain.

Aspect 15. A quantity of mCLs according to any one of aspects 1-13, wherein the mCLs are T cells are classical T cells (alpha beta receptor expressing), T cells expressing gamma/delta TCRs, CD8 and CD4 phenotypes, MAIT T cells restricted to MR-1 non-classical HLA, killer T cells or killer innate-like T cells.

Aspect 16. A composition comprising a quantity of cells, wherein the quantity of cells comprises mCLs according to any one of aspects 1-15, and wherein the percentage of cells that are mCLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%,
optionally wherein the composition is a pharmaceutical composition suitable for administration into a human subject.

Aspect 17. An admixture comprising
(i) a quantity of mCLs according to any one of aspects 1-15 or a composition according to aspect 16; and
(ii) a quantity of a homodimer comprising two heterodimers, wherein each heterodimer comprises:
a) a first polypeptide comprising:
   i) a human papillomavirus epitope, wherein the epitope comprises the amino acid sequence YMLDLQPETT (SEQ ID NO:1) or YMLDLQPET (SEQ ID NO:2); and
   ii) a first major histocompatibility complex (MHC) polypeptide, wherein the first MHC polypeptide is a β2-microglobulin (β2M) polypeptide comprising an amino acid sequence as depicted in FIG. 4B, and
b) a second polypeptide comprising:
   i) two copies of an IL-2 polypeptide, each copy comprising an amino acid sequence as depicted in FIG. 3B;
   iii) a second MHC polypeptide, wherein the second MHC polypeptide is an MHC class I heavy chain polypeptide comprising an amino acid sequence as depicted in FIG. 3C; and
   iv) an immunoglobulin (Ig) Fc polypeptide.
wherein the first polypeptide and the second polypeptide are covalently linked to one another via a disulfide bond.

Aspect 18. An admixture according to aspect 17,
wherein the Ig Fc polypeptide comprises an amino acid sequence having at least about 95% percent amino acid sequence identity to the amino acid sequence depicted in FIG. 3D, wherein the percent sequence identity is determinable by a sequence alignment performed using BLAST, and wherein the Ig Fc polypeptide comprises a Leu at position 14 and a Leu at position 15 based on the amino acid numbering depicted FIG. 3D,
wherein the first and second polypeptides are covalently linked to one another via a disulfide bond between the Cys residue at amino acid 12 of the β2M polypeptide and the Cys residue at amino acid 236 of the class I MHC heavy chain polypeptide,
wherein the first polypeptide comprises a peptide linker between the epitope and the β2M polypeptide, and wherein the second polypeptide comprises a peptide linker between one or more of:
a) a first copy of the IL-2 polypeptide and a second copy of the IL-2 polypeptide;
b) one of the two copies of the IL-2 polypeptide and the MHC class I heavy chain polypeptide; and
c) the MHC class I heavy chain polypeptide and the Ig Fc polypeptide,
optionally wherein the homodimer comprises two heterodimers, wherein each heterodimer comprises a first polypeptide comprising the amino acid sequence set forth in SEQ ID NO:14, and a second polypeptide comprising the amino acid sequence set forth in SEQ ID NO:15 or SEQ ID NO:18.

Aspect 19. A method of making a quantity of mCLs according to any one of aspects 1-15, wherein the method comprises modifying a quantity of cells by introducing one or more nucleic acids encoding
(i) an antigen-binding domain specific for a cancer-associated antigen, and
(ii) an HPV E7_{11-19/20} TCR.

Aspect 20. A method according to aspect 19, wherein the method comprises the step of at least partially separating target cells comprising an HPV E7_{11-19/20} TCR, optionally wherein the step of at least partially separating target cells comprising an HPV E7_{11-19/20} TCR comprises binding the target cells to an HPV16 E7 peptide comprising the amino acid sequence YMLDLQPETT (SEQ ID NO:1) or YMLDLQPET (SEQ ID NO:2).

Aspect 21. A method according to aspect 20, wherein the HPV16 E7 peptide is immobilized on an insoluble support.

Aspect 22. A method according to aspect 21, wherein insoluble support is a bead.

Aspect 23. A method according to aspect 19, wherein the HPV16 peptide is a peptide-loaded MHC class I multimer.

Aspect 24. A method according to any one of aspects 19-23, wherein mCLs comprise a CAR, and wherein the CAR comprises: a) an extracellular domain comprising the antigen-binding domain; b) a transmembrane region; and c) a cytoplasmic domain comprising an intracellular signaling domain.

Aspect 25. A method according to aspect 24, wherein the intracellular signaling domain comprises a signaling domain from the zeta chain of human CD3.

Aspect 26. A method according to aspect 24 or 25, wherein the cytoplasmic domain comprises one or more costimulatory polypeptides.

Aspect 27. A method according to aspect 26, wherein the costimulatory polypeptide is selected from CD28, 4-1BB, and OX-40.

Aspect 28. A method according to any one of aspects 19-27, wherein the antigen-binding domain is a single-chain Fv polypeptide or a nanobody.

Aspect 29. A method according to any one of aspects 19-28, wherein mCLs comprise a CAR, and wherein the CAR is a single polypeptide chain CAR.

Aspect 30. A method according to any one of aspects 19-28, wherein mCLs comprise a CAR, and wherein the CAR comprises two polypeptide chains.

Aspect 31. A method according to any one of aspects 19-30, wherein the cancer-associated antigen is selected from AFP, BCMA, CD10, CD117, CD123, CD133, CD128, CD171. CD19, CD20, CD22, CD30, CD33, CD34, CD38, CD5, CD56, CD7, CD70, CD80, CD86, CEA, CLD18, CLL-1, cMet, EGFR, EGFRVIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

Aspect 32. A method according to any one of aspects 19-31, wherein the percentage of total number of cells in the composition that are target mCLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

Aspect 33. A method according to any one of aspects 19-32, wherein prior to step (ii), the composition comprising a quantity of cells is contacted, in vitro or in vivo, with a TMP, wherein the TMP comprises a heterodimeric polypeptide comprising:
a) a first polypeptide comprising: (i) an HPV16 peptide comprising the amino acid sequence YMLDLQPETT (SEQ ID NO:1) or YMLDLQPET (SEQ ID NO:2); (ii) a linker; and (iii) a β2 microglobulin polypeptide; and
b) a second polypeptide comprising: (i) two copies of an IL-2 polypeptide; (ii) an MHC class I heavy chain polypeptide; and (iii) an immunoglobulin (Ig) Fc polypeptide,
optionally wherein the TMP comprises a homodimer comprising two heterodimeric polypeptides, wherein each heterodimeric polypeptide comprises a first polypeptide comprising the amino acid sequence set forth in SEQ ID NO:14, and a second polypeptide comprising the amino acid sequence set forth in SEQ ID NO: 15 or SEQ ID NO:18.

Aspect 34. A method according to any one of aspects 19-33, wherein at least 50% of the target cells are CD8+ T cells.

Aspect 35. A method according to any one of aspects 19-34, comprising, between steps (i) and (ii), enriching the cells for CD8+ T cells.

Aspect 36. A method according to any one of aspects 19-35, comprising, between steps (ii) and (iii), enriching the cells for CD8+ T cells.

Aspect 37. A method of treating a cancer in an individual, the method comprising introducing into the individual a composition comprising a quantity of modified cytotoxic cells according to any one of aspects 1-15, or a pharmaceutical composition prepared according to the method of any one of aspects 16-33.

Aspect 38. A method of according to aspect 37, further comprising administering to the individual a composition comprising a TMP comprising either:
a) heterodimeric polypeptide comprising
a first polypeptide comprising: (i) an HPV16 peptide comprising the amino acid sequence YMLDLQPETT (SEQ ID NO:1) or YMLDLQPET (SEQ ID NO:2); (ii) a linker; and (iii) a β2 microglobulin polypeptide; and
a second polypeptide comprising: (i) two copies of an IL-2 polypeptide; (ii) an MHC class I heavy chain polypeptide; and (iii) a variant immunoglobulin (Ig) Fc polypeptide,
optionally wherein the TMP comprises a homodimer comprising two heterodimeric polypeptides, wherein each heterodimeric polypeptide comprises a first polypeptide comprising the amino acid sequence set forth in SEQ ID NO:14, and a second polypeptide comprising the amino acid sequence set forth in SEQ ID NO:15 or SEQ ID NO:18;
or
b) a single-chain polypeptide comprising: (i) an HPV16 peptide comprising the amino acid sequence YMLDLQPETT (SEQ ID NO:1) or YMLDLQPET (SEQ ID NO:2), (ii) a β2 microglobulin polypeptide, (iii) one or more copies of a variant IL-2 polypeptide; (iv) an MHC class I heavy chain polypeptide, and (v) a variant immunoglobulin (Ig) Fc polypeptide.

Aspect 39. A method according to aspect 37 or aspect 38, wherein said administering a composition comprising a quantity of genetically modified cytotoxic cells comprises administering a quantity of genetically modified cytotoxic cells that is equal to or less than a number selected from the group consisting of 10 cells/kg body weight, 10² cells/kg body weight, 10³ cells/kg body weight, 10⁺ cells/kg body weight, 10⁵ cells/kg body weight, 10⁶ cells/kg body weight, 10⁷ cells/kg body weight, 10⁸ cells/kg body weight and 10⁹ cells/kg body weight.

Aspect 40. A method according to any of aspects 37-39, wherein said administering a composition comprising a quantity of genetically modified cytotoxic cells comprises administering a quantity of genetically modified cytotoxic cells that is equal to or less than 10⁷ cells/kg body weight.

Aspect 41. A method according to any of aspects 37-40, wherein the individual does not undergo a lymphodepleting regimen prior to the introducing step.

Aspect 42. A method according to any one of aspects 37-41, wherein said administering is intramuscular, intravenous, peritumoral, or intratumoral.

Aspect 43. A method according to any one of aspects 37-42, further comprising administering one or more checkpoint inhibitors to the individual.

Aspect 44. A method according to aspect 43, wherein the checkpoint inhibitor is an antibody that binds to a polypeptide selected from the group consisting of CD27, CD28, CD40, CD122, CD96, CD73, CD47, OX40, GITR, CSFIR, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1, and PD-L2.

Aspect 45. A method according to aspect 43, wherein the checkpoint inhibitor is an antibody specific for PD-1, PD-L1, or CTLA4.

Aspect 46. A method according to aspect 45, wherein the one or more checkpoint inhibitors is selected from the group consisting of nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, Mogamulizumab, Varlilumab, Avelumab, Galiximab, AMP-514, AUNP 12, Indoximod, NLG-919, INCB024360, KN035, and combinations thereof.

### EXAMPLES

The following example is put forth so as to provide those of ordinary skill in the art with a more complete disclosure and is not intended to limit the scope of what the inventors regard as their invention nor are they intended to represent that the experiments below are all or the only experiments performed.

### Example 1

A population of CAR-T cells are modified in vitro by the addition of one or more nucleic acids encoding an HPV E7_{11 19/20} TCR to prepare a composition of mCTLs. Alternatively, T cells are first modified by the addition of nucleic acids encoding an HPV E7_{11-19/20} TCR, and thereafter modified by the addition of one or more nucleic acids encoding a CAR, to prepare a composition comprising a population of mCTLs. The population of mCTLs is increased in vitro, e.g., using a recombinantly produced IL-2 such as Proleukin® or a TMP such as CUE-101 prior to administration to a patient.

The patient does not receive lymphodepleting therapy prior to administration of the mCTLs. The number of mCTLs administered to the patient are at least one order of magnitude lower than the dose that would be in the absence of administration of the TMP. Following administration of the mCTLs, the patient is administered one or more doses of CUE-101 in an amount up to about 4 mg/kg body weight in order to increase the number of mCTLs to a desired range and thereafter to maintain the number of mCTLs within a desired range. Following administration of the mCTLs, the patient does not receive a wild-type IL-2 such as Proleukin®. Following administration of the mCTLs, the patient does not experience severe symptoms associated with cytokine release syndrome.

### Example 2

Example 2 is carried out the same as Example 1, except that the patient is administered a dose of CUE-101 in an amount up to about 4 mg/kg body weight prior to administration of the mCTLs. Following administration of the mCTLs, the patient is administered one or more additional doses of CUE-101 in an amount up to about 4 mg/kg body weight in order to increase the number of mCTLs to a desired range and/or maintain the number of mCTLs within a desired range. Following administration of the mCTLs, the patient does not receive a wild-type IL-2 such as Proleukin®. Following administration of the mCTLs, the patient does not experience severe symptoms associated with cytokine release syndrome.

### Example 3

A composition of mCTLs is prepared comprising Yescarta® CAR-T cells that include nucleic acids encoding an HPV E7_{11-19/20} TCR.

The patient does not receive lymphodepleting therapy prior to administration of the mCTLs. Optionally, prior to administration of the mCTLs to the patient, the patient is administered a dose of CUE-101 in an amount up to about 4 mg/kg body weight.

The number of Yescarta® CAR-T cells administered to the patient are up to about 2×10⁵ CAR-positive viable T cells per kg body weight up to a maximum of 2×10⁷ CAR-positive viable T cells, optionally up to about 2×10⁴ CAR-positive viable T cells per kg body weight up to a maximum of 2×10⁶ CAR-positive viable T cells. Following administration of the mCTLs, the patient is administered additional doses of CUE-101 in an amount up to about 4 mg/kg body weight in order to increase the number of mCTLs to a desired range, and thereafter to maintain the number of mCTLs within a desired range. Following administration of the mCTLs, the patient does not receive a wild-type IL-2 such as Proleukin®. Following administration of the mCTLs, the patient does not experience severe symptoms associated with cytokine release syndrome.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A quantity of modified cytotoxic T cells (“mCTLs”), wherein the mCTLs comprise: a) one or more nucleic acids comprising nucleotide sequences encoding a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2); and b) one or more nucleic acids comprising nucleotide sequences encoding a chimeric antigen receptor (CAR), wherein the CAR comprises an antigen-binding domain specific for a cancer-associated antigen, and wherein the percentage of target mCTLs cells in the composition exceeds at least 1% of the total number of T cells in the composition.

2. A quantity of modified cytotoxic T cells (“mCTLs”), wherein the mCTLs comprise: a) a T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2); and b) a chimeric antigen receptor (CAR), wherein the CAR comprises an antigen-binding domain specific for a cancer-associated antigen, and wherein the percentage of target mCTLs cells in the composition exceeds at least 1% of the total number of T cells in the composition.

3. A quantity of modified cytotoxic T cells (“mCTLs”), wherein the mCTLs comprise: a) a heterologous T-cell receptor (TCR) specific for MHC class I polypeptides that present a human papilloma virus (HPV) E7 peptide comprising the amino acid sequence YMLDLQPETT (SEQ ID NO:1) or YMLDLQPET (SEQ ID NO:2); and b) a chimeric antigen receptor (CAR), wherein the CAR comprises an antigen-binding domain specific for a cancer-associated antigen.

4. A quantity of mCTLs according to any one of claims 1-3, wherein the CAR comprises: a) an extracellular domain comprising the antigen-binding domain; b) a transmembrane region; and c) a cytoplasmic domain comprising an intracellular signaling domain.

5. A quantity of mCTLs according to claim 4, wherein the cytoplasmic domain comprises one or more costimulatory polypeptides.

6. A quantity of mCTLs according to claim 5, wherein the costimulatory polypeptide is selected from CD28, 4-1BB, and OX-40.

7. A composition according to any one of claims 4-6, wherein the intracellular signaling domain comprises a signaling domain from the zeta chain of human CD3.

8. A quantity of mCTLs according to any one of claims 1-7, wherein the antigen-binding domain is a single-chain Fv polypeptide or a nanobody.

9. A quantity of mCTLs according to any one of claims 1-8, wherein the CAR is a single polypeptide chain CAR.

10. A quantity of mCTLs according to any one of claims 1-8, wherein the CAR comprises at least two polypeptide chains.

11. A quantity of mCTLs according to any one of claims 1-10, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRVIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2.

12. A quantity of mCTLs according to any one of claims 1-11, wherein the TCR comprises: (i) an α chain having at least 90% amino acid sequence identity to the α-chain amino acid sequence depicted in FIG. 2A; and (ii) a having at least 90% amino acid sequence identity to the β chain amino acid sequence depicted in FIG. 2B.

13. A quantity of mCTLs according to any one of claims 1-11, wherein the TCR comprises: (i) an α chain having at least 90% amino acid sequence identity to the α-chain amino acid sequence depicted in FIG. 2C; and (ii) a having at least 90% amino acid sequence identity to the β chain amino acid sequence depicted in FIG. 2D.

14. A quantity of mCTLs according to any one of claims 1-13, wherein the mCTLs are CD8+ T cells.

15. A composition comprising a quantity of T cells, wherein the quantity of T cells comprises mCTLs according to any one of claims 1-13, and wherein the percentage of T cells that are mCTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

16. A composition according to claim 15, wherein the composition is a pharmaceutical composition suitable for administration into a human subject.

17. An admixture comprising
(i) a quantity of mCTLs according to any one of claims 1-14 or a composition according to claim 15 or 16; and
(ii) a quantity of a homodimer comprising two heterodimers, wherein each heterodimer comprises:
a) a first polypeptide comprising:
i) a human papillomavirus epitope, wherein the epitope comprises the amino acid sequence YMLDLQPETT (SEQ ID NO:1) or YMLDLQPET (SEQ ID NO:2); and
ii) a first major histocompatibility complex (MHC) polypeptide, wherein the first MHC polypeptide is a β2-microglobulin (β2M) polypeptide comprising an amino acid sequence as depicted in FIG. 4B, and
b) a second polypeptide comprising:
i) two copies of an IL-2 polypeptide, each copy comprising an amino acid sequence as depicted in FIG. 3B;
iii) a second MHC polypeptide, wherein the second MHC polypeptide is an MHC class I heavy chain polypeptide comprising an amino acid sequence as depicted in FIG. 3C; and
iv) an immunoglobulin (Ig) Fc polypeptide,
wherein the first polypeptide and the second polypeptide are covalently linked to one another via a disulfide bond.

18. An admixture according to claim 17,
wherein the Ig Fc polypeptide comprises an amino acid sequence having at least about 95% percent amino acid sequence identity to the amino acid sequence depicted in FIG. 3D, wherein the percent sequence identity is determinable by a sequence alignment performed using BLAST, and wherein the Ig Fc polypeptide comprises a Leu at position 14 and a Leu at position 15 based on the amino acid numbering depicted FIG. 3D,
wherein the first and second polypeptides are covalently linked to one another via a disulfide bond between the Cys residue at amino acid 12 of the β2M polypeptide and the Cys residue at amino acid 236 of the class I MHC heavy chain polypeptide,
wherein the first polypeptide comprises a peptide linker between the epitope and the β2M polypeptide, and wherein the second polypeptide comprises a peptide linker between one or more of:
a) a first copy of the IL-2 polypeptide and a second copy of the IL-2 polypeptide;
b) one of the two copies of the IL-2 polypeptide and the MHC class I heavy chain polypeptide; and
c) the MHC class I heavy chain polypeptide and the Ig Fc polypeptide.

19. A method of making a quantity of mCTLs according to any one of claims 1-14, wherein the method comprises modifying a quantity of T cells by introducing one or more nucleic acids encoding
(i) a chimeric antigen receptor (CAR) that comprises an antigen-binding domain specific for a cancer-associated antigen, and
(ii) an HPV E7_{11-19/20} TCR.

20. A method according to claim 19, wherein the method comprises the step of at least partially separating target T cells comprising an HPV E7_{11-19/20} TCR, optionally wherein the step of at least partially separating target T cells comprising an HPV E7_{11-19/20} TCR comprises binding the target T cells to an HPV16 E7 peptide comprising the amino acid sequence YMLDLQPETT (SEQ ID NO:1) or YMLDLQPET (SEQ ID NO:2).

21. A method according to claim 20, wherein the HPV16 E7 peptide is immobilized on an insoluble support.

22. A method according to claim 21, wherein insoluble support is a bead.

23. A method according to claim 19, wherein the HPV16 E7 peptide is a peptide-loaded MHC class I multimer.

24. A method according to any one of claims 19-23, wherein the CAR comprises: a) an extracellular domain comprising the antigen-binding domain; b) a transmembrane region; and c) a cytoplasmic domain comprising an intracellular signaling domain.

25. A method according to claim 24, wherein the intracellular signaling domain comprises a signaling domain from the zeta chain of human CD3.

26. A method according to claim 24 or 25, wherein the cytoplasmic domain comprises one or more costimulatory polypeptides.

27. A method according to claim 26, wherein the costimulatory polypeptide is selected from CD28, 4-1BB, and OX-40.

28. A method according to any one of claims 19-27, wherein the antigen-binding domain is a single-chain Fv polypeptide or a nanobody.

29. A method according to any one of claims 19-28, wherein the CAR is a single polypeptide chain CAR.

30. A method according to any one of claims 19-28, wherein the CAR comprises two polypeptide chains.

31. A method according to any one of claims 19-30, wherein the cancer-associated antigen is selected from AFP, BCMA, CD10, CD117, CD123, CD133, CD128, CD171, CD19, CD20, CD22, CD30, CD33, CD34, CD38, CD5, CD56, CD7, CD70, CD80, CD86, CEA, CLD18, CLL-1, cMet, EGFR, EGFRVIII, EpCAM, EphA2, GD-2, glypican-3, GPC3, HER-2, kappa immunoglobulin, LeY, LMP1, mesothelin, MG7, MUC1, NKG2D ligand, PD-L1, PSCA, PSMA, ROR1, ROR1R, TACI, and VEGFR2, optionally wherein the CAR comprises a scFv that binds CD19 or BCMA.

32. A method according to any one of claims 19-31, wherein the percentage of total number of T cells in the composition that are target CTLs is selected from the group consisting of at least 10%, at least 20%, at least 30%, at least 40%, at least 50%, at least 60%, at least 70%, at least 80%, at least 90%, at least 95%, at least 96%, at least 97%, at least 98%, at least 99%, and 100%.

33. A method according to any one of claims 19-32, wherein prior to step (ii), the composition comprising a quantity of T cells is contacted, in vitro or in vivo, with a composition comprising a T cell modulatory polypeptide (TMP) comprising:
a) a first polypeptide comprising: (i) an HPV16 peptide comprising the amino acid sequence YMLDLQPETT (SEQ ID NO:1) or YMLDLQPET (SEQ ID NO:2); (ii) a linker; and (iii) a β2 microglobulin polypeptide; and
b) a second polypeptide comprising: (i) two copies of an IL-2 polypeptide; (ii) a major histocompatibility complex class I (MHC class I) heavy chain polypeptide; and (iii) an immunoglobulin (Ig) Fc polypeptide.

34. A method according to any one of claims 19-33, wherein at least 50% of the target T cells are CD8+ T cells.

35. A method according to any one of claims 19-34, comprising, between steps (i) and (ii), enriching the T cells for CD8+ T cells.

36. A method according to any one of claims 19-35, comprising, between steps (ii) and (iii), enriching the T cells for CD8+ T cells.

37. A method of treating a cancer in an individual, the method comprising introducing into the individual a composition comprising a quantity of modified cytotoxic T cells according to any one of claims 1-15, or a pharmaceutical composition prepared according to the method of any one of claims 16-33.

38. A method of according to claim 37, further comprising administering to the individual a composition comprising a TMP comprising:
a) a first polypeptide comprising: (i) an HPV16 peptide comprising the amino acid sequence YMLDLQPETT (SEQ ID NO1) or YMLDLQPET (SEQ ID NO:2); (ii) a linker; and (iii) a β2 microglobulin polypeptide; and
b) a second polypeptide comprising: (i) two copies of an IL-2 polypeptide; (ii) a major histocompatibility complex class I (MHC class I) heavy chain polypeptide; and (iii) an immunoglobulin (Ig) Fc polypeptide,
optionally wherein the homodimer comprises two heterodimers, wherein each heterodimer comprises a first polypeptide comprising the amino acid sequence set forth in SEQ ID NO:14, and a second polypeptide comprising the amino acid sequence set forth in SEQ ID NO:15 or SEQ ID NO:18.

39. A method according to claim 37 or claim 38, wherein said administering a composition comprising a quantity of genetically modified cytotoxic T cells comprises administering a quantity of genetically modified cytotoxic T cells that is equal to or less than a number selected from the group consisting of 10 cells/kg body weight, 10² cells/kg body weight, 10³ cells/kg body weight, 10⁴ cells/kg body weight, 10⁵ cells/kg body weight, 10⁶ cells/kg body weight, 10⁷ cells/kg body weight, 10⁸ cells/kg body weight and 10⁹ cells/kg body weight.

40. A method according to any of claims 37-39, wherein said administering a composition comprising a quantity of genetically modified cytotoxic T cells comprises administering a quantity of genetically modified cytotoxic T cells that is equal to or less than 10⁷ cells/kg body weight.

41. A method according to any of claims 37-40, wherein the individual does not undergo a lymphodepleting regimen prior to the introducing step.

42. A method according to any one of claims 37-41, wherein said administering is intramuscular, intravenous, peritumoral, or intratumoral.

43. A method according to any one of claims 37-42, further comprising administering one or more checkpoint inhibitors to the individual.

44. A method according to claim 43, wherein the checkpoint inhibitor is an antibody that binds to a polypeptide selected from the group consisting of CD27, CD28, CD40, CD122, CD96, CD73, CD47, OX40, GITR, CSFIR, JAK, PI3K delta, PI3K gamma, TAM, arginase, CD137, ICOS, A2AR, B7-H3, B7-H4, BTLA, CTLA-4, LAG3, TIM3, VISTA, CD96, TIGIT, CD122, PD-1, PD-L1, and PD-L2.

45. A method according to claim 43, wherein the checkpoint inhibitor is an antibody specific for PD-1, PD-L1, or CTLA4.

46. A method according to claim 45, wherein the one or more checkpoint inhibitors is selected from the group consisting of nivolumab, pembrolizumab, pidilizumab, AMP-224, MPDL3280A, MDX-1105, MEDI-4736, arelumab, ipilimumab, tremelimumab, pidilizumab, IMP321, MGA271, BMS-986016, lirilumab, urelumab, PF-05082566, IPH2101, MEDI-6469, CP-870,893, Mogamulizumab, Varlilumab, Avclumab, Galiximab, AMP-514, AUNP 12, Indoximod, NLG-919, INCB024360, KN035, and combinations thereof.


SEQUENCE LISTING (144 sequences)

SEQ ID NO: 1 - protein, 10 aa, synthetic construct
YMLDLQPETT

SEQ ID NO: 2 - protein, 9 aa, synthetic construct
YMLDLQPET

SEQ ID NO: 3 - protein, 99 aa, synthetic construct [sequence omitted]

SEQ ID NO: 4 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 5 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 6 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 275 aa, synthetic construct [sequence omitted]

SEQ ID NO: 8 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 9 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 231 aa, synthetic construct
AAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 5 aa, synthetic construct
GGGGS

SEQ ID NO: 12 - protein, 6 aa, synthetic construct
AAAAGG

SEQ ID NO: 13 - protein, 15 aa, synthetic construct
GGGGSGGGGSGGGGS

SEQ ID NO: 14 - protein, 124 aa, synthetic construct
YMLDLQPETTGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 15 - protein, 812 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 232 aa, synthetic construct
AAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 813 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 242 aa, synthetic construct
AVTLDCTYDTSDPSYGLFWYKQPSSGEMIFLIYQGSYDQQNATEGRYSLNFQKARKSANLVISASQLGDSAMYFCAMREDSLSGGYQKVTFGIGTKLQVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - protein, 275 aa, synthetic construct
VFLECVQDMDHENMFWYRQDPGLGLRLIYFSYDVKMKEKGDIPEGYSVSREKKERFSLILESASTNQTSMYLCASSLLSRASYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 21 - protein, 236 aa, synthetic construct
VTLSCTYDTSENNYYLFWYKQPPSRQMILVIRQEAYKQQNATENRFSVNFQKAAKSFSLKISDSQLGDTAMYFCARNNAGNMLTFGGGTRLMVKPHIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 271 aa, synthetic construct
LTVTCSQNMNHEYMSWYRQDPGLGLRQIYYSMNVEVTDKGDVPEGYKVSRKEKRNFPLILESPSPNQTSLYFCASRRTGSQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 23 - protein, 20 aa, synthetic construct
GGGGSGGGGSGGGGSGGGGS

SEQ ID NO: 24 - protein, 5 aa, synthetic construct
AAAGG

SEQ ID NO: 25 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 107 aa, synthetic construct [sequence omitted]

SEQ ID NO: 28 - protein, 120 aa, synthetic construct [sequence omitted]

SEQ ID NO: 29 - protein, 50 aa, synthetic construct
  VARIANT 6..50: note = Wherein up to 9 copies of GGGGS may be omitted.
GGGGSGGGGSGGGGSGGGGSGGGGSGGGGSGGGGSGGGGSGGGGSGGGGS

SEQ ID NO: 30 - protein, 11 aa, synthetic construct
RASQDVNTAVA

SEQ ID NO: 31 - protein, 6 aa, synthetic construct
SASFLY

SEQ ID NO: 32 - protein, 8 aa, synthetic construct
QQHYTTPP

SEQ ID NO: 33 - protein, 8 aa, synthetic construct
GFNIKDTY

SEQ ID NO: 34 - protein, 8 aa, synthetic construct
IYPTNGYT

SEQ ID NO: 35 - protein, 13 aa, synthetic construct
SRWGGDGFYAMDY

SEQ ID NO: 36 - protein, 242 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 37 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 107 aa, synthetic construct [sequence omitted]

SEQ ID NO: 40 - protein, 119 aa, synthetic construct [sequence omitted]

SEQ ID NO: 41 - protein, 11 aa, synthetic construct
KASQDVSIGVA

SEQ ID NO: 42 - protein, 6 aa, synthetic construct
SASYRY

SEQ ID NO: 43 - protein, 8 aa, synthetic construct
QQYYIYPY

SEQ ID NO: 44 - protein, 10 aa, synthetic construct
GFTFTDYTMD

SEQ ID NO: 45 - protein, 18 aa, synthetic construct
ADVNPNSGGSIYNQRFKG

SEQ ID NO: 46 - protein, 12 aa, synthetic construct
ARNLGPSFYFDY

SEQ ID NO: 47 - protein, 15 aa, synthetic construct
KASQSVDYDGDSYLN

SEQ ID NO: 48 - protein, 7 aa, synthetic construct
DASNLVS

SEQ ID NO: 49 - protein, 9 aa, synthetic construct
QQSTEDPWT

SEQ ID NO: 50 - protein, 5 aa, synthetic construct
SYWMN

SEQ ID NO: 51 - protein, 17 aa, synthetic construct
QIWPGDGDTNYNGKFKG

SEQ ID NO: 52 - protein, 15 aa, synthetic construct
RETTTVGRYYYAMDY

SEQ ID NO: 53 - protein, 249 aa, synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVS

SEQ ID NO: 54 - protein, 217 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTESS

SEQ ID NO: 55 - protein, 450 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 107 aa, synthetic construct
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTK

SEQ ID NO: 57 - protein, 120 aa, synthetic construct
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 58 - protein, 14 aa, synthetic construct
TGTSSDIGGYNSVS

SEQ ID NO: 59 - protein, 11 aa, synthetic construct
LMIYGVNNRPS

SEQ ID NO: 60 - protein, 10 aa, synthetic construct
SSYDIESATP

SEQ ID NO: 61 - protein, 10 aa, synthetic construct
GYSFTSYWIG

SEQ ID NO: 62 - protein, 16 aa, synthetic construct
WMGIIDPGDSRTRYSP

SEQ ID NO: 63 - protein, 11 aa, synthetic construct
GQLYGGTYMDG

SEQ ID NO: 64 - protein, 244 aa, synthetic construct
QVQLQQSGAEVKKPGASVKVSCKASGYTFTGYYMHWVRQAPGQGLEWMGRINPNSGGTNYAQKFQGRVTMTRDTSISTAYMELSRLRSEDTAVYYCARGRYYGMDVWGQGTMVTVSSGGGGSGGGGSGGGGSGGGGSEIVLTQSPATLSLSPGERATISCRASQSVSSNFAWYQQRPGQAPRLLIYDASNRATGIPPRFSGSGSGTDFTLTISSLEPEDFAAYYCHQRSNWLYTFGQGTKVDIK

SEQ ID NO: 65 - protein, 253 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDLRRTVVTPRAYYGMDVWGQGTTVTVSSGGGGSGGGGSGGGGSGGGGSDIQLTQSPSTLSASVGDRVTITCQASQDISNSLNWYQQKAGKAPKLLIYDASTLETGVPSRFSGSGSGTDFSFTISSLQPEDIATYYCQQHDNLPLTFGQGTKVEIK

SEQ ID NO: 66 - protein, 217 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLGQPKAAPSVTLFPPSSEELQANKATLVCLISDFYPGAVTVAWKADSSPVKAGVETTTPDSKQSNNKYAASSYLSLTPEQWKSHRSYSCQVTHEGSTVEKTVAPTECS

SEQ ID NO: 67 - protein, 453 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 111 aa, synthetic construct
QSVLTQPPSASGTPGQRVTISCSGSSSNIGSNTVNWYQQLPGTAPKLLIFNYHQRPSGVPDRFSGSKSGSSASLAISGLQSEDEADYYCAAWDDSLNGWVFGGGTKLTVLG

SEQ ID NO: 69 - protein, 131 aa, synthetic construct
EVQLVESGGGLVKPGGSLRLSCAASGFTFGDYALSWFRQAPGKGLEWVGVSRSKAYGGTTDYAASVKGRFTISRDDSKSTAYLQMNSLKTEDTAVYYCASSGYSSGWTPFDYWGQGTLVTVSSASTKGPSV

SEQ ID NO: 70 - protein, 8 aa, synthetic construct
SSNIGSNT

SEQ ID NO: 71 - protein, 11 aa, synthetic construct
AAWDDSLNGWV

SEQ ID NO: 72 - protein, 8 aa, synthetic construct
GFTFGDYA

SEQ ID NO: 73 - protein, 10 aa, synthetic construct
SRSKAYGGTT

SEQ ID NO: 74 - protein, 14 aa, synthetic construct
ASSGYSSGWTPFDY

SEQ ID NO: 75 - protein, 249 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 76 - protein, 249 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSS

SEQ ID NO: 77 - protein, 11 aa, synthetic construct
SASQDISNYLN

SEQ ID NO: 78 - protein, 7 aa, synthetic construct
YTSNLHS

SEQ ID NO: 79 - protein, 9 aa, synthetic construct
QQYRKLPWT

SEQ ID NO: 80 - protein, 5 aa, synthetic construct
NYWMH

SEQ ID NO: 81 - protein, 17 aa, synthetic construct
ATYRGHSDTYYNQKFKG

SEQ ID NO: 82 - protein, 12 aa, synthetic construct
GAIYNGYDVLDN

SEQ ID NO: 83 - protein, 108 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 84 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 85 - protein, 20 aa, synthetic construct
VTSAPDTRPAPGSTAPPAHG

SEQ ID NO: 86 - protein, 43 aa, synthetic construct
SNIKFRPGSVVVQLTLAFREGTINVHDVETQFNQYKTEAASRY

SEQ ID NO: 87 - protein, 35 aa, synthetic construct
SVVVQLTLAFREGTINVHDVETQFNQYKTEAASRY

SEQ ID NO: 88 - protein, 20 aa, synthetic construct
LAFREGTINVHDVETQFNQY

SEQ ID NO: 89 - protein, 25 aa, synthetic construct
SNIKFRPGSVVVQLTLAAFREGTIN

SEQ ID NO: 90 - protein, 5 aa, synthetic construct
RYGMS

SEQ ID NO: 91 - protein, 17 aa, synthetic construct
TISGGGTYIYYPDSVKG

SEQ ID NO: 92 - protein, 13 aa, synthetic construct
DNYGRNYDYGMDY

SEQ ID NO: 93 - protein, 10 aa, synthetic construct
SATSSVSYIH

SEQ ID NO: 94 - protein, 7 aa, synthetic construct
STSNLAS

SEQ ID NO: 95 - protein, 9 aa, synthetic construct
QQRSSSPFT

SEQ ID NO: 96 - protein, 5 aa, synthetic construct
GYAMS

SEQ ID NO: 97 - protein, 17 aa, synthetic construct
TISSGGTYIYYPDSVKG

SEQ ID NO: 98 - protein, 12 aa, synthetic construct
LGGDNYYEYFDV

SEQ ID NO: 99 - protein, 15 aa, synthetic construct
RASKSVSTSGYSYMH

SEQ ID NO: 100 - protein, 7 aa, synthetic construct
LASNLES

SEQ ID NO: 101 - protein, 9 aa, synthetic construct
QHSRELPFT

SEQ ID NO: 102 - protein, 5 aa, synthetic construct
DYAMN

SEQ ID NO: 103 - protein, 17 aa, synthetic construct
VISTFSGNINFNQKFKG

SEQ ID NO: 104 - protein, 10 aa, synthetic construct
SDYYGPYFDY

SEQ ID NO: 105 - protein, 16 aa, synthetic construct
RSSQTIVHSNGNTYLE

SEQ ID NO: 106 - protein, 7 aa, synthetic construct
KVSNRFS

SEQ ID NO: 107 - protein, 9 aa, synthetic construct
FQGSHVPFT

SEQ ID NO: 108 - protein, 9 aa, synthetic construct
LGGDNYYEY

SEQ ID NO: 109 - protein, 15 aa, synthetic construct
TASKSVSTSGYSYMH

SEQ ID NO: 110 - protein, 7 aa, synthetic construct
LVSNLES

SEQ ID NO: 111 - protein, 10 aa, synthetic construct
QHIRELTRSE

SEQ ID NO: 112 - protein, 8 aa, synthetic construct
GFTFSNYY

SEQ ID NO: 113 - protein, 8 aa, synthetic construct
ISGRGSTI

SEQ ID NO: 114 - protein, 10 aa, synthetic construct
VKDRGGYSPY

SEQ ID NO: 115 - protein, 6 aa, synthetic construct
QSISTY

SEQ ID NO: 116 - protein, 10 aa, synthetic construct
QQSYSTPPIT

SEQ ID NO: 117 - protein, 5 aa, Homo sapiens
DKTHT

SEQ ID NO: 118 - protein, 4 aa, Homo sapiens
CPPC

SEQ ID NO: 119 - protein, 15 aa, Homo sapiens
CPEPKSCDTPPPCPR

SEQ ID NO: 120 - protein, 12 aa, Homo sapiens
ELKTPLGDTTHT

SEQ ID NO: 121 - protein, 10 aa, Homo sapiens
KSCDKTHTCP

SEQ ID NO: 122 - protein, 7 aa, Homo sapiens
KCCVDCP

SEQ ID NO: 123 - protein, 7 aa, Homo sapiens
KYGPPCP

SEQ ID NO: 124 - protein, 15 aa, Homo sapiens
EPKSCDKTHTCPPCP

SEQ ID NO: 125 - protein, 12 aa, Homo sapiens
ERKCCVECPPCP

SEQ ID NO: 126 - protein, 17 aa, Homo sapiens
ELKTPLGDTTHTCPRCP

SEQ ID NO: 127 - protein, 12 aa, Homo sapiens
SPNMVPHAHHAQ

SEQ ID NO: 128 - protein, 45 aa, synthetic construct
TTTPAPRPPTPAPTIASQPLSLRPEACRPAAGGAVHTRGLDFACD

SEQ ID NO: 129 - protein, 24 aa, synthetic construct
IYIWAPLAGTCGVLLLSLVITLYC

SEQ ID NO: 130 - protein, 23 aa, synthetic construct
LGLLVAGVLVLLVSLGVAIHLCC

SEQ ID NO: 131 - protein, 25 aa, synthetic construct
ALIVLGGVAGLLLFIGLGIFFCVRC

SEQ ID NO: 132 - protein, 23 aa, synthetic construct
LCYLLDGILFIYGVILTALFLRV

SEQ ID NO: 133 - protein, 26 aa, synthetic construct
WVLVVVGGVLACYSLLVTVAFIIFWV

SEQ ID NO: 134 - protein, 26 aa, synthetic construct
VAAILGLGLVLGLLGPLAILLALYLL

SEQ ID NO: 135 - protein, 24 aa, synthetic construct
ALPAALAVISFLLGLGLGVACVLA

SEQ ID NO: 136 - protein, 42 aa, synthetic construct
KRGRKKLLYIFKQPFMRPVQTTQEEDGCSCRFPEEEEGGCEL

SEQ ID NO: 137 - protein, 44 aa, synthetic construct
FWVRSKRSRLLHSDYMNMTPRRPGPTRKHYQPYAPPRDFAAYRS

SEQ ID NO: 138 - protein, 35 aa, synthetic construct
TKKKYSSSVHDPNGEYMFMRAVNTAKKSRLTDVTL

SEQ ID NO: 139 - protein, 37 aa, synthetic construct
RRDQRLPPDAHKPPGGGSFRTPIQEEQADAHSTLAKI

SEQ ID NO: 140 - protein, 114 aa, synthetic construct
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 141 - protein, 49 aa, synthetic construct
HQRRKYRSNKGESPVEPAEPCRYSCPREEEGSTIPIQEDYRKPEPACSP

SEQ ID NO: 142 - protein, 187 aa, synthetic construct
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 143 - protein, 54 aa, synthetic construct
HIWQLRSQCMWPRETQLLLEVPPSTEDARSCQFPEEERGERSAEEKGRLGDLWV

SEQ ID NO: 144 - protein, 60 aa, synthetic construct
CVKRRKPRGDVVKVIVSVQRKRQEAEGEATVIEALQAPPDVTTVAVEETIPSFTGRSPNH